SEQUENCE LISTING (24 sequences)

SEQ ID NO: 1 - protein, 1245 aa, Bacillus thuringiensis
MATINELYPVPYNVLAHPIKEVDDPYSWSNLLKGIQEGWEEWGKTGQKKLFEDHLTIAWNLYKTGKLDYEALTKASISLIGFIPGAEAAVPFINMEVDFVWPKLFGANTEGKDQQLFNAIMDAVNKMVDNKFLSYNLSTLNKTIEGIQGNLGLFQNAIQVAICQGSTPERVNFDQNCTPCNPNQPCKDDLDRVASRFDTANSQFTQHLPEFKNPWSDENSTQEFKRTSVELTLPMYTTVATLHLLLYEGYIEFMTKWNFHNEQYLNNLKVELQQLIHSYSETVRTSFLQFLPTLNNRSKSSVNAYNRYVRNMTVNCLDIAATWPTFDTHNYHQGGKLDLTRIILSDTAGPIEEYTTGDKTSGPEHSNITPNNILDTPSPTYQHSFVSVDSIVYSRKELQQLDIATYSTNNSNNCHPYGLRLSYTDGSRYDYGDNQPDFTTSNNNYCHNSYTAPITLVNARHLYNAKGSLQNVESLVVSTVNGGSGSCICDAWINYLRPPQTAKNESFPDQKDNVLYPITETVNKGTGGNLGVISAYVPMELVPENVIGDVNADTKLPLTQLKGFPFEKYGSEYNNRGISLVREWINGNNAVKLSNSQSVGIQITNQTEQKYEIRCRYASKGDNNVYFNVDLSENPFRNSISFGSTESSVVGVQGENGKYILKSITTVEIPAGSFYVHITNQGSSDLFLDRIEFVPKIQFQFCDNNNLHCDCNNPVDTDCTFCCVCTSLTDCDCNNPRGLDCTLCCQVENQLPSFVTLTDLQNITTQVNALVASSEHDTLATDVSDYEIEEVVLKVDALSGEVFGKEKKALRKLVNHTKRLSKARNLLIGGNFDNLDAWYRGRNVVNVSDHELFKSDHYLLPPPTLYSSYMFQKVEESKLKANTRYSVSGFIAHAEDLEIVVSEYGQEVKKVVQVPYGEAFPLTSRGAICCPPRSTSNGKPADPHEFSYSIDVGTLDVEANPGIELGLRIVERTGMARYSNLEIREDPPLKKNELRNVQRAARNWRSAYDQERAEVTALIQPVLNQINALYENEDWNGAIRSGVSYHDLEAIVLPTLPKLNHWFMSDMLGEQGSILAQFQEALDRAYTQLEESTILHNGHFTTDAANWTIEGDAHHAILEDGRRVLRLPDWSSSVSQTIEIENFDPDKEYQLVFHAQGEGTVSLQHGEEGEYVETHPHKSANFTTSHRQGLTFETNKVTVEITSEDGEFLVDHIALVEAPLPTDDQSSDGNTFSNTNSNTSMNNNQ

SEQ ID NO: 2 - protein, 803 aa, Bacillus thuringiensis
MTCQLQAQPLIPYNVLAGYPTSNTGSPIGNAGNQFDQFEQTVKELKEAWEAFQKNGSFSLAALEKGFDAAIGGGSFDYLGLVQAGLGLVGTLGAAIPGVSVAVPLISMLVGVFWPKGTNNQENLITVIDKEVQRILDEKLSDQLIKKINADLNAFTDLVTRLEEVIIDATFENHKPVLQVSKSNYMKVDSAYFSTGGILTLGMSDFLTDTYSKLTFPLYVLGATMKLSAYHSYIQFGNTWLNKVYDLSSDEGKTMSQALARAKQHMRQDIAFYTSQALNMFTGNLPSLSSNKYAINDYNVYTRAMVLNGLDIVATWPTLYPDDYSSQIKLEKTRVIFSDMVGQSESRDGSVTIKNIFDNTDSHQHGSIGLNSISYFPDELQKAQLRMYDYNHKPYCTDCFCWPYGVILNYNKNTFRYGDNDPGLSGDVQLPAPMSVVNAQTQTAQYTDGENINTDTGRSWLCTLRGYCTTNCFPGRGCYNNSTGYGESCNQSLPGQKIHALYPFTQTNVLGQSGKLGLLASHIPYDLSPNNTIGDKDTDSTNIVAKGIPVEKGYASSGQKVEIIREWINGANVVQLSPGQSWGMDFTNSTGGQYMVRCRYASTNDTPIFFNLVYDGGSNPIYNQMTFPATKETPAHDSVDNEILGIKGINGNYSLMNVKDSVELPSGKFHVFFTNNGSSAIYLDRLEFVPLDQPAAPTQSTQPINYPITSRLPHRSGEPPAIIWEKSGNVRGNQLTISAQGVPENSQIYLSVGGDRQILDRSNGFKLVNYSPTYSFTNIQASSSNLVDITSGTITGQVQVSNL

SEQ ID NO: 3 - protein, 1186 aa, Bacillus thuringiensis
MDCNLQSQQNIPYNVLAIPVSNVNALVDTAGDLKKAWEEFQKTGSFSLTALQQGFSASQGGAFNYLTLLQSGISLAGSFVPGGTFVAPIVNMVIGWLWPHENKTADTENLIKLIDEEIQKQLNKALLDQDRNNWTSFLESIFDTSATVSNADIDAQWSGTVDTTNRQQKTPTTSDYLNVVGKFDSADSSIITNENQIMNGNFDVAAAPYFVDGATLRLSLYQSYIKFCNSWIDAVGFSTNDANTQKANLARTKLTMRSTINEYTQRVMKVFKDSKNMPTIGTNKFSVDAYNVYVKGMTLNVLDMVAIWSSLYPNDYTSQTAIEQTRVTFSNMVGQEEGTDGTLKIYNTFDSLSYQHSLIPNNNVNLISYYTDELQNLELAVYTPKGGSGYAYPYGFILNYANSNYKYGDNDPTGKPLNKQDGPIQQINAATQNSKYLDGETINGIGASLPGYCTTGSSATEQPFSCTGTANSYKASCNPSDTNQKINALYAFTQTNVKGSTGKLGVLASLVPYDLNPKNVFGELDSDTNNVILKGIPAEKGYFPNNARPTVVKEWINGASAVPFYSGNTLFMTATNLTATQYKIRIRYANPNSDTQISVLITQNGSQISNSNLTLYSTTDSSMSSNLPQNVYVTGENGNYTLLDLYSTTNVLSTGDITLKLTGGNQKIFIDRIEFIPTMPVPAPTNNTNNNNGDNGNNNPPHHGCAIAGTQQLCSGPPKFEQVSDLEKITTQVYMLFKSSSYEELAIKVSSYQINQVALKVMALSDEKFCEEKRLLRKLVNKANQLLEARNLLVGGNFETTQNWVLGTNAYDNYDSFLFNGNYLSLQPASGFFTSYAYQKIDESTLKPYTRYKVSGFIGQSNQVELIISRYGKEIDKILNVPYAGPLPITADASITCCAPEIDQCDGGQSDSHFFNYSIDVGALHPELNPGDEIGLKIVQSNGYISISNLEIIEERPLTEMEIQAVNRKDQKWKRELLLECASVSELLQPIDNQIDSLFKDANWYNDILPHVTYQTLKNIIVPDLPKLKHWFIDHLPGEYHEIEQKMKEALKHAFTQLDEKNLIHNGHFATNLIDWQVEGDAFMKVLENNALALQLSNWDSSVSQSIDILEFDEDKAYKLRVYAQGSGTIQFGNCEDEAIQFNTNSFVYKEKIIYFDSPSINLHIQSEGSEFVVSSIDLVELSDDE

SEQ ID NO: 4 - protein, 1167 aa, Bacillus thuringiensis
MTNPTILYPSYHNVLAHPIRLDSFFDPFVETFKDLKGAWEEFGKTGYMDPLKQHLQIAWDTSQNGTVDYLALTKASISLIGLIPGADAVVPFINMFVDFIFPKLFGRGSQQNAQAQFFELIIEKVKELVDEDFRNFTLNNLLNYLDGMQTALSHFQNDVQIAICQGEQPGLMLDQTPTACTPTTDHLISVRESFKDARTTIETALPHFKNPNLSTNDNTPDFNSDTVLLTLPMYTTGATLNLILHQGYIQFAERWKSVNYDESFINQTKVDLQRRIQDYSTTVSTTFEKFKPTLNPSNKESVNKYNRYVRSMTLQSLDIAATWPTLDNVNYPSNVDIQLDQTRLVFSDVAGPWEGNDNITSNIIDVLTPINTGIGFQESSDLRKFTYPRIELQSMQFHGQYVNSKSVEHCYSDGLKLNYKNKTITAGVSNIDESNQNNKHNYGPVINSPITDINVNSQNSQYLDLNSVMVNGGQKVTGCSPLSSNGNSNNAALPNQKINVIYSVQSNDKPEKHADTYRKWGYMSSHIPYDLVPENVIGDIDPDTKQPSLLLKGFPAEKGYGDSIAYVSEPLNGANAVKLTSYQVLQMEVTNQTTQKYRIRIRYATGGDTAASIWFHIIGPSGNDLTNEGHNFSSVSSRNKMFVQGNNGKYVLNILTDSIELPSGQQTILIQNTNSQDLFLDRIEFISLPSTSTPTSTNFVEPESLEKIINQVNQLFSSSSQTELAHTVSDYKIDQVVLKVNALSDDVFGVEKKALRKLVNQAKQLSKARNVLVGGNFEKGHEWALSREATMVANHELFKGDHLLLPPPTLYPSYAYQKIDESKLKSNTRYTVSGFIAQSEHLEVVVSRYGKEVHDMLDIPYEEALPISSDESPNCCKPAACQCSSCDGSQSDSHFFSYSIDVGSLQSDVNLGIEFGLRIAKPNGFAKISNLEIKEDRPLTEKEIKKVQRKEQKWKKAFNQEQAEVATTLQPTLDQINALYQNEDWNGSVHPASDYQHLSAVVVPTLPKQRHWFMEGREGEHVVLTQQFQQALDRAFQQIEEQNLTHNGNLANGLTDWTVTGDAQLTIFDEDPVLELAHWDASISQTIEIMDFEGRHRIQTACTWKRQRNSYRSTWRKRLETMTFNTTSFTTQEQTFYFEGDTVDVHVQSENNTFLIDSVELIEIIEE

SEQ ID NO: 5 - protein, 1167 aa, Bacillus thuringiensis
MTNPTILYPSYHNVLAHPIRLDSFFDPFVETFKDLKSAWEEFGKTGYMDPLKQHLQIAWDTSQNGTVDYLALTKASISLIGLIPGADAVVPFINMFVDFIFPKLFGRGSQQNAQAQFFELIIEKVKELVDEDFRNFTLNNLLNYLDGMQTALSHFQNDVQIAICQGEQPGLMLDQTPTACTPTTDHLISVRESFKDARTTIETALPHFKNPNLSTNDNTPDFNSDTVLLTLPMYTTAATLNLILHQGYIQFAERWKSVNYDESFINQTKVDLQRRIQDYSTTVSTTFEKFKPTLNPSNKESVNKYNRYVRSMTLQSLDIAATWPTLDNVNYPSNVDIQLDQTRLVFSDVAGPWEGNDNITSNIIDVLTPINTGIGFQESSDLRKFTYPRIELQSMQFHGQYVNSKSVEHCYSDGLKLNYKNKTITAGVSNIDESNQNNKHNYGPVINSPITDINVNSQNSQYLDLNSVMVNGGQKVAGCSPLSSNGNSNNAALPNQKINVIYSVQSNDKPEKHADTYRKWGYMSSHIPYDLVPENVIGDIDPDTKQPSLLLKGFPAEKGYGDSIAYVSEPLNGANAVKLTSYQVLKMEVTNQTTQKYRIRIRYATGGDTAASIWFHIIGPSGNDLTNEGHNFSSVSSRNKMFVQGNNGKYVLNILTDSIELPSGQQTILIQNTNSQDLFLDRIEFISLPSTSTPTSTNFVEPESLEKIINQVNQLFSSSSQTELAHTVSDYKIDQVVLKVNALSDDVFGVEKKALRKLVNQAKQLSKARNVLVGGNFEKGHEWALSREATMVANHELFKGDHLLLPPPTLYPSYAYQKIDESKLKSNTRYTVSGFIAQSEHLEVVVSRYGKEVHDMLDIPYEEALPISSDESPNCCKPAACQCSSCDGSQSDSHFFSYSIDVGSLQSDVNLGIEFGLRIAKPNGFAKISNLEIKEDRPLTEKEIKKVQRKEQKWKKAFNQEQAEVATTLQPTLDQINALYQNEDWNGSVHPHVTYQHLSAVVVPTLPKQRHWFMEDREGEHVVLTQQFQQALDRAFQQIEEQNLIHNGNFANGLTDWTVTGDAQLSIFDEDPVLELAHWDASISQTIEIMDFEEDTEYKLRVRGKGKGTVTVQHGEEELETMTFNSTSFTTQEQTFYFEGDTVDVHVQSENNTFLIDSVELIEIIEE

SEQ ID NO: 6 - protein, 475 aa, Bacillus thuringiensis
MIIDSKTTLPRHSLIHTIKLNSNKKYGPGDMTNGNQFIISKQEWATIGAYIQTGLGLPVNEQQLRTHVNLSQDISIPSDFSQLYDVYCSDKTSAEWWNKNLYPLIIKSANDIASYGFKVAGDPSIKKDGYFKKLQDELDNIVDNNSDDDAIAKAIKDFKARCGILIKEAKQYEEAAKNIVTSLDQFLHGDQKKLEGVINIQKRLKEVQTALNQAHGESSPAHKELLEKVKNLKTTLERTIKAEQDLEKKVEYSFLLGPLLGFVVYEILENTAVQHIKNQIDEIKEQLDSAQHDLDRDVKIIGMLNSINTDIDNLYSQGQEAIKVFQKLQGIWATIGAQIENLRTTSLQEVQDSDDADEIQIELEDASDAWLVVAQEARDFTLNAYSTNSRQNLPINVISDSCNCSTTNMTSNQYSNPTTNMTSNQYMISHEYTSLPNNFMLSRNSNLEYKCPENNFMIYWYNNSDWYNNSDWYNN

SEQ ID NO: 7 - protein, 10 aa, synthetic construct
  REGION 1..10: note = Description of Artificial Sequence: Synthetic peptide
VYIDRIEFVP

SEQ ID NO: 8 - protein, 10 aa, synthetic construct
  REGION 1..10: note = Description of Artificial Sequence: Synthetic peptide
VYIDKIEFIP

SEQ ID NO: 9 - protein, 10 aa, synthetic construct
  REGION 1..10: note = Description of Artificial Sequence: Synthetic peptide
VLIDKIEFLP

SEQ ID NO: 10 - protein, 10 aa, synthetic construct
  REGION 1..10: note = Description of Artificial Sequence: Synthetic peptide
VFLDRIEFIP

SEQ ID NO: 11 - protein, 10 aa, synthetic construct
  REGION 1..10: note = Description of Artificial Sequence: Synthetic peptide
LFLDRIEFVP

SEQ ID NO: 12 - protein, 10 aa, synthetic construct
  REGION 1..10: note = Description of Artificial Sequence: Synthetic peptide
FYVDSIEFIP

SEQ ID NO: 13 - protein, 10 aa, synthetic construct
  REGION 1..10: note = Description of Artificial Sequence: Synthetic peptide
VYIDRIEFIP

SEQ ID NO: 14 - protein, 10 aa, synthetic construct
  REGION 1..10: note = Description of Artificial Sequence: Synthetic peptide
VYVDRIEFIP

SEQ ID NO: 15 - protein, 10 aa, synthetic construct
  REGION 1..10: note = Description of Artificial Sequence: Synthetic peptide
IYIDKIEFIP

SEQ ID NO: 16 - protein, 10 aa, synthetic construct
  REGION 1..10: note = Description of Artificial Sequence: Synthetic peptide
MVLDRIEFVP

SEQ ID NO: 17 - protein, 10 aa, synthetic construct
  REGION 1..10: note = Description of Artificial Sequence: Synthetic peptide
IYLDRLEFVP

SEQ ID NO: 18 - protein, 10 aa, synthetic construct
  REGION 1..10: note = Description of Artificial Sequence: Synthetic peptide
IFIDRIEFIP

SEQ ID NO: 19 - protein, 10 aa, synthetic construct
  REGION 1..10: note = Description of Artificial Sequence: Synthetic peptide
LILDKIEFLP

SEQ ID NO: 20 - protein, 10 aa, synthetic construct
  REGION 1..10: note = Description of Artificial Sequence: Synthetic peptide
FVLDKIELIP

SEQ ID NO: 21 - protein, 10 aa, synthetic construct
  REGION 1..10: note = Description of Artificial Sequence: Synthetic peptide
LFLDRIEFIS

SEQ ID NO: 22 - protein, 9 aa, synthetic construct
  REGION 1..9: note = Description of Artificial Sequence: Synthetic peptide
IIDKIEFIP

SEQ ID NO: 23 - protein, 5 aa, synthetic construct
  REGION 1..5: note = Description of Artificial Sequence: Synthetic peptide
DRIEF

SEQ ID NO: 24 - protein, 5 aa, synthetic construct
  REGION 1..5: note = Description of Artificial Sequence: Synthetic peptide
DRLEF

(19) United States
(12) Patent Application Publication
(10) Pub. No.: US 2024/0148800 A1
(43) Pub. Date: May 9, 2024

(54) PURIFIED ANTHELMINTIC COMPOSITIONS AND RELATED METHODS

(71) Applicant: UNIVERSITY OF MASSACHUSETTS, Boston, MA (US)

(72) Inventors: Raffi Van Aroian, Worcester, MA (US); Gary R. Ostroff, Worcester, MA (US)

(21) Appl. No.: 18/485,796

(22) Filed: Oct. 12, 2023

Related U.S. Application Data
(62) Division of application No. 16/607,677, filed on Oct. 23, 2019, now Pat. No. 11,844,815.
(60) Provisional application No. 62/510,081, filed on May 23, 2017.

(51) Int. Cl. A61K 35/741; A61K 9/48; A61K 38/16; A61P 33/10

(52) U.S. Cl. CPC A61K 35/741; A61K 9/48; A61K 38/164; A61P 33/10; A61K 45/06

(57) ABSTRACT

Compositions and methods for treating or reducing the severity of occurrence of a parasitic worm or helminth infection in a subject are described. The methods include administering to the subject a therapeutically effective amount of a composition comprising isolated native, bioactive nematicidal crystals formed from a single type of nematicidal crystal protein. The isolated native, bioactive nematicidal crystals are substantially free of any bacterial spores or host bacterial proteins, other than nematicidal crystal protein in the form of a crystal. Methods for making isolated native, bioactive nematicidal crystals are also described. The crystal proteins may be full length, truncated, variant, or sub-variant Cry proteins. Examples of crystal proteins include Cry5B, Cry21, Cry14A, Cry6A, and Cry13A.

### RELATED APPLICATIONS

This application is a division of U.S. patent application Ser. No. 16/607,677, filed Oct. 23, 2019, which is a 35 U.S.C. § 371 filing of International Patent Application No. PCT/US2018/033962, filed May 22, 2018, which claims priority to U.S. Provisional Patent Application Ser. No. 62/510,081, filed May 23, 2017, the entire contents of which are incorporated herein by reference.

### STATEMENT OF FEDERALLY SPONSORED RESEARCH

This invention was made with government support under Grant Nos. AI056189 awarded by the United States Department of Health and Human Services (HHS) National Institutes of Health (NIH), and NIFA 2016-67015-24861 awarded by the United States Department of Agriculture (USDA) National Institutes of Food and Agriculture (NIFA). The government has certain rights in this invention.

### SEQUENCE LISTING

The instant application contains a Sequence Listing which has been submitted electronically in XML format and is hereby incorporated by reference in its entirety. Said XML file, created on Oct. 12, 2023, is named 746913_UM9-222USDIV2_ST26.xml and is 35,238 bytes in size.

### BACKGROUND

Soil-transmitted helminthes (STHs) that parasitize the GI tract of humans infect 2.3 billion of the poorest peoples and >400,000,000 of the poorest children worldwide. Crystal (Cry) proteins made by the soil bacterium Bacillus thuringiensis (Bt) are candidate agents that provide safe and effective treatment of STHs. However, despite the established anthelmintic biological activity of Cry proteins, significant challenges remain with respect to effective delivery of intact, biologically active Cry proteins into the gastrointestinal (GI) tract of humans and animals for treating STHs. The cost and scalability of Cry protein expression and purification limits its application as a practical STH therapy in the developing world where treatments must be available at a very low cost (less than $1/dose) and in very large quantities to treat a large and poor patient population.

A cheap, simple, and scalable way to deliver Cry proteins is to express it in B. thuringiensis, which is ideally suited to express very high levels of Cry protein and which is already fermented cheaply on a massive scale for environmental release. However, production of high levels of Cry protein in Bt requires sporulation. Thus, the Cry protein compositions currently deployed into the environment are in the form of a “spore-crystal lysate” (SCL) that includes both the crystal protein and spores from the bacterium. Use of these SCL compositions in humans is problematic due to the presence of bacterial spores which can contain many of the enterotoxin genes that causes food poisoning in humans. Furthermore, the inclusion of Bt spores makes formulation for administration to humans and animals both more difficult, as spores are difficult to work with, and less efficient, since gram-for-gram any formulation would necessarily include a significant amount of inactive ingredient (spore) along with active ingredient (crystal). Separation of spores from crystal proteins is difficult as the two are very similar in size.

Accordingly, there remains an urgent need in the art for new approaches to delivering protein therapeutics such as anthelmintic proteins to the GI tract.

### SUMMARY

The instant disclosure is based on the discovery that a sporulation defective or sporulation incompetent bacterium can be employed for production of highly purified preparations of nematicidal crystals suitable for pharmaceutical use. The purified nematicidal crystal preparations of the invention offer superior anti-helminthic properties to crystal proteins purified from spore crystal lysate (SCL). Moreover, such preparations are substantially free of contaminating spores and host bacterial proteins which are unsuitable for administration to humans. In certain exemplary embodiments, the preparations are substantially free of soluble cell components, lipids, and cell wall debris.

In one aspect, the instant disclosure provides a pharmaceutical composition comprising an isolated native, bioactive nematicidal crystal formed from a single type of nematicidal crystal protein. The nematicidal crystal protein is produced by a non-sporulating form of host bacterium, and the pharmaceutical composition is substantially free of any bacterial spores or host bacterial proteins other than nematicidal crystal protein in the form of a crystal.

In certain embodiments, the pharmaceutical composition includes excipients suitable for oral administration to a human subject. In certain embodiments, the host bacterium is a Bacillus species. In some embodiments, the host bacterium is a Bacillus thuringiensis (Bt). In some embodiments, the host bacterium is an Escherichia coli or Pseudomonas fluorescens species.

In some embodiments, the non-sporulating host bacterium is genetically engineered to have a genetic mutation that results in a defect in sporulation such that the native, bioactive nematicidal crystal is trapped in the cytosol of the bacterium. In certain embodiments, the genetic mutation resulting in a defect of sporulation is the deletion or inactivation of one or more genes selected from the group consisting of: kinA, kinB, spo0A, spo0B, spo0E, spo0F, spo0J, spo0M spoIIB, spoIID, spoIIE, spoIIF, spoIIG, spoIIL, spoIIM, spoIIIA, spoIIIB, spoIIIE, spoIVA, spoIVC, spoIVD, spoVG, spoVK, spoVL, spoVM, spoVN, spoVP, spoVQ, spoVID, σH, σF, σE, σG, and σK. In one embodiment, the genetic mutation resulting in a defect of sporulation is the deletion or inactivation of the spo0A gene.

In some embodiments, the host bacterium is genetically engineered to express the single type of nematicidal crystal protein under the control of a non-sporulation specific promoter. In certain embodiments, the non-sporulation specific promoter is a Cry3A, GerA, GNAT, or TadA promoter. In certain embodiments, the single type of nematicidal crystal protein is selected from the group consisting of Cry5B, Cry5C, Cry5D, Cry6A, Cry13A, Cry14A, Cry21A, Cry21B, Cry 55B, and variants and truncations thereof. In certain embodiments, the nematicidal crystal protein is Cry5B, or variants or fragments thereof. In some embodiments, the nematicidal crystal protein is Cry5B variant Ser407Cys.

In certain embodiments of the pharmaceutical compositions disclosed herein, the pharmaceutical compositions further comprise a second crystal protein in the form of an isolated native, bioactive nematicidal crystal formed from only the second crystal protein.

In some embodiments, the pharmaceutical composition comprises at least 95% isolated native, bioactive nematicidal crystal content.

In some embodiments the isolated native, bioactive nematicidal crystals are in an orally-available dosage form. In some embodiments, the pharmaceutical composition is in a dry powdered form and is encapsulated by a pharmaceutical capsule.

In another aspect, the disclosure provides a method for producing a pharmaceutical composition of the invention, the method comprising: (a) growing a non-sporulating form of a host bacterium that is genetically engineered to express a single type of nematicidal crystal protein, wherein the non-sporulating host bacterium produces native, bioactive nematicidal crystals formed from the single type of nematicidal crystal protein, wherein the host bacterium is grow in a growth medium, and optionally wherein the non-sporulating host bacterium releases the native, bioactive nematicidal crystals into the growth medium; and (b) isolating and concentrating the native, bioactive nematicidal crystals to form isolated, native, bioactive nematicidal crystals.

In some embodiments of this method, the host bacterium is a Bacillus species. In some embodiments, the host bacterium is a Bacillus thuringiensis (Bt). In some embodiments, the host bacterium is an E. coli or P. fluorescens species.

In some embodiments of the methods, the non-sporulating host bacterium is genetically engineered to have a genetic mutation that results in a defect in sporulation such that the native, bioactive nematicidal crystal is trapped in the cytosol of the bacterium. In some embodiments, the genetic mutation resulting in a defect of sporulation is the deletion or inactivation of one or more genes selected from the group consisting of: kinA, kinB, spo0A, spo0B, spo0E, spo0F, spo0J, spo0M spoIIB, spoIID, spoIIE, spoIIF, spoIIG, spoIIL, spoIIM, spoIIIA, spoIIIB, spoIIIE, spoIVA, spoIVC, spoIVD, spoVG, spoVK, spoVL, spoVM, spoVN, spoVP, spoVQ, spoVID, σH, σF, σE, σG, and σK. In some embodiments, the genetic mutation resulting in a defect of sporulation is the deletion or inactivation of the spo0A gene.

In certain embodiments of the method above, the host bacterium is genetically engineered to express the single type of nematicidal crystal protein under the control of a non-sporulation specific promoter. In certain embodiments, the non-sporulation specific promoter is a Cry3A, GerA, GNAT, or TadA promoter. In certain embodiments, the single type of nematicidal crystal protein is selected from the group consisting of Cry5B, Cry5C, Cry5D, Cry6A, Cry13A, Cry14A, Cry21A, Cry21B, Cry55B, and variants and truncations thereof. In certain embodiments, the nematicidal crystal protein is Cry5B, or variants or fragments thereof. In some embodiments, the nematicidal crystal protein is Cry5B variant Ser407Cys.

In certain embodiments, the method further comprises a step of exposing the non-sporulating host bacterium to an antimicrobial compound to inactivate the host bacterium. In certain embodiments, the antimicrobial compound is iodine. In certain embodiments, the antimicrobial compound is a pharmaceutical antibiotic. In certain embodiments, the antimicrobial compound is a beta-lactam antibiotic. In certain embodiments, the antimicrobial compound is an organic solvent selected form the group consisting of a terpene, hexane, and formaldehyde. In certain embodiments, the antimicrobial compound is a terpene. In certain embodiments, the terpene is selected from the group consisting of thymol, eugenol, geraniol, carvacrol, and citral, or a combination thereof. In certain embodiments, the terpene is carvacrol. In certain embodiments, the antimicrobial compound is hexane.

In certain embodiments, the method includes a step of adding a food-grade oil to the inactivated host bacterium to extract the inactivating agent from the nematicidal crystal protein. In certain embodiments, the food-grade oil is selected from the group consisting of corn oil, soybean oil, coconut oil, cottonseed oil, olive oil, palm oil, peanut oil, rapeseed oil, safflower oil, and sunflower oil. In some embodiments, the food-grade oil is corn oil. In some embodiments, the food-grade oil is corn oil. In certain embodiments, the method includes a step of a step of adding an organic solvent to the inactivated host bacterium to extract cell components from the nematicidal crystal protein. In certain embodiments, the organic solvent is hexane. In certain embodiments, the hexane is added to the inactivated host bacterium to 50% v/v. In certain embodiments, the method includes a step of centrifuging the mixture of organic solvent and inactivated host bacterium to pellet the nematicidal crystal protein.

In certain embodiments, the method further includes a step of homogenizing the inactivated host bacterium to form a bacterial lysate that includes the native, bioactive nematicidal crystals. In some embodiments, the method further includes a step of concentrating the bacterial lysate. In certain embodiments, the step of concentrating the bacterial lysate is selected from the group consisting of centrifugation, ultrafiltration, and diafiltration.

In some embodiments, the method further comprises a step of formulating the isolated native, bioactive nematicidal crystals in an orally-available dosage form. In some embodiments, the step of formulating comprises lyophilizing or spray-drying the isolated native, bioactive nematicidal crystals. In some embodiments, the step of formulating comprises encapsulating the isolated native, bioactive nematicidal crystals in a pharmaceutical-grade capsule.

In still another aspect, the disclosure provides a method for producing a pharmaceutical composition, including: (a) growing a non-sporulating form of a host bacterium that is engineered to express a single type of nematicidal crystal protein, wherein the non-sporulating host bacterium produces native bioactive nematicidal crystals formed from the single type of nematicidal crystal protein; (b) inactivating the grown non-sporulating host bacterium by exposing the grown non-sporulating host bacterium to an antimicrobial compound; (c) homogenizing the inactivated non-sporulating host bacterium to form a bacterial lysate; and (d) concentrating the native bioactive nematicidal crystals in the bacterial lysate to form isolated native, bioactive nematicidal crystals.

In some embodiments, this method further comprises a step of concentrating the grown nonsporulating host bacterium before inactivating the grown non-sporulating host bacterium. In some embodiments, the host bacterium is a Bacillus species. In some embodiments, the host bacterium is a Bacillus thuringiensis (Bt). In certain embodiments, the host bacterium is an E. coli or P. fluorescens species.

In some embodiments of this method, the non-sporulating host bacterium is genetically engineered to have a genetic mutation that results in a defect in sporulation such that the native, bioactive nematicidal crystal is trapped in the cytosol of the bacterium. In some embodiments, the genetic mutation resulting in a defect of sporulation is the deletion or inactivation of one or more genes selected from the group consisting of: kinA, kinB, spo0A, spo0B, spo0E, spo0F, spo0J, spo0M spoIIB, spoIID, spoIIE, spoIIF, spoIIG, spoIIL, spoIIM, spoIIIA, spoIIIB, spoIIIE, spoIVA, spoIVC, spoIVD, spoVG, spoVK, spoVL, spoVM, spoVN, spoVP, spoVQ, spoVID, σH, σF, σE, σG, and σK. In certain embodiments, the genetic mutation resulting in a defect of sporulation is the deletion or inactivation of the spo0A gene.

In some embodiments of the method, the host bacterium is genetically engineered to express the single type of nematicidal crystal protein under the control of a non-sporulation specific promoter. In certain embodiments, the non-sporulation specific promoter is a Cry3A, GerA, GNAT, or TadA promoter. In certain embodiments, the single type of nematicidal crystal protein is selected from the group consisting of Cry5B, Cry5C, Cry5D, Cry6A, Cry13A, Cry14A, Cry21A, Cry21B, Cry55B, and variants and truncations thereof. In certain embodiments, the nematicidal crystal protein is Cry5B, or variants or fragments thereof. In some embodiments, the nematicidal crystal protein is Cry5B variant Ser407Cys.

In some embodiments of the methods, the antimicrobial compound is iodine. In other embodiments of the methods, the antimicrobial compound is a pharmaceutical antibiotic. In some embodiments of the methods, the antimicrobial compound is a beta-lactam antibiotic. In some embodiments, the antimicrobial compound is an organic solvent selected from the group consisting of a terpene, hexane, or formaldehyde. In certain embodiments, the antimicrobial compound is a terpene. In certain embodiments, the terpene is selected from the group consisting of thymol, eugenol, geraniol, carvacrol, and citral, or a combination thereof. In certain embodiments, the terpene is carvacrol. In certain embodiments, the antimicrobial compound is hexane.

In some embodiments, the method further comprises a step of extracting the antimicrobial compound from the inactivated host bacterium. In certain embodiments, the method includes a step of adding a food-grade oil to the inactivated host bacterium to extract the inactivating agent from the nematicidal crystal protein. In certain embodiments, the food-grade oil is selected from the group consisting of corn oil, soybean oil, coconut oil, cottonseed oil, olive oil, palm oil, peanut oil, rapeseed oil, safflower oil, and sunflower oil. In some embodiments, the food-grade oil is corn oil. In certain embodiments, a step of adding an organic solvent to the inactivated host bacterium to extract cell components from the nematicidal crystal protein. In certain embodiments, the organic solvent is hexane. In certain embodiments, the hexane is added to the inactivated host bacterium to 50% v/v. In certain embodiments, the method includes a step of centrifuging the mixture of organic solvent and inactivated host bacterium to pellet the nematicidal crystal protein.

In some embodiments, the method includes a step of formulating the isolated native, bioactive nematicidal crystals in an orally-available dosage form. In some embodiments, the method includes the step of formulating comprises lyophilizing or spray drying the isolated native, bioactive nematicidal crystals. In some embodiments, the step of formulating comprises encapsulating the isolated purified native, bioactive nematicidal crystals in a pharmaceutical-grade capsule.

In yet another aspect, the disclosure provides a method of treating a parasitic worm infection in a subject comprising: administering to the subject a therapeutically effective amount of a pharmaceutical composition comprising isolated native, bioactive nematicidal crystals formed from a single type of nematicidal crystal protein, wherein the pharmaceutical composition does not contain bacterial spores.

In some embodiments of the method of treating, the nematicidal crystal protein is selected from the group consisting of Cry5B, Cry5C, Cry5D, Cry6A, Cry13A, Cry14A, Cry21A, Cry21B, Cry55B, and variants and truncations thereof. In some embodiments, the nematicidal crystal protein is Cry5B. In some embodiments, the nematicidal crystal protein is Cry5B variant Ser407Cys.

In some embodiments of the method of treating, the composition further comprises a second crystal protein in the form of an isolated native, bioactive nematicidal crystal formed from only the second crystal protein. In some embodiments, the pharmaceutical composition comprises at least about 95% isolated native, bioactive nematicidal crystal. In some embodiments, the composition is in a dry powdered form and is encapsulated by a pharmaceutical capsule.

### BRIEF DESCRIPTION OF THE DRAWINGS

FIGS. 1A-1B are diagrams showing conserved blocks of amino acids in wild-type crystal proteins. FIG. 1A is depicts the positions of conserved blocks among certain Cry proteins. de Maagd, R. A., et al. “How Bacillus thuringiensis has evolved specific toxins to colonize the insect world.” Trends in Genetics 17(4): 193-99, 195 (FIG. 2a) (April 2001). FIG. 1B illustrates the positions of conserved blocks among certain Cry proteins. Schnepf, E., et al. “Bacillus thuringiensis and Its Pesticidal Crystal Proteins.” Microbiology and Molecular Biology Reviews 62(3): 775-806, 781 (FIG. 3) (September 1998).

FIG. 2 illustrates the amino acid sequence of Cry5Ba1 [SEQ ID NO:1].

FIG. 3 illustrates the amino acid sequence of Cry13Aa1 [SEQ ID NO:2].

FIG. 4 illustrates the amino acid sequence of Cry14Aa1 [SEQ ID NO:3].

FIGS. 5A-5C show amino acid sequences of other crystal proteins. FIG. 5A illustrates the amino acid sequence of Cry21Aa1 [SEQ ID NO:4]. FIG. 5B illustrates the amino acid sequence of Cry21Aa2 (98% identical to Cry21Aa1) [SEQ ID NO:5]. FIG. 5C illustrates the amino acid sequence of Cry6A [SEQ ID NO:6].

FIG. 6 is a flow chart showing the steps for purifying nematicidal crystal protein from inactivated BaCC (IBaCC).

FIG. 7 is a picture of a protein gel with samples from several steps of the purifying process for Cry5B PCC.

FIG. 8 is a phase-contrast photomicrograph of Cry5B PCC at 1000× magnification.

FIG. 9A shows a graph of changes in whipworm motility over time following in vitro exposure to doses of Cry5B PCC. FIG. 9B shows a bar histogram of in vivo whipworm burdens in hamsters treated with Cry5B PCC and water control.

FIG. 10A shows a graph of changes in hookworm motility over time following in vitro exposure to doses of Cry5B PCC. FIG. 10B shows a histogram of hookworm burdens in hamsters treated with Cry5B PCC and water control. FIG. 10C shows a bar histogram of hookworm burdens in hamsters before and after treatment with Cry5B PCC and water control.

FIG. 11A shows a plot of hookworm burden in hamsters following treatment with either Cry5B spore-crystal lysate (SCL) or water control. FIG. 11B shows a plot of eggs per gram in hamsters following treatment with either SCL or water control.

FIG. 12 shows a bar histogram of various concentrations of Cry5B PCC toxicity on Ancyclostoma ceylanicum hookworm eggs; some of the Cry5B PCC was treated by the addition of lysozyme.

FIG. 13A shows a schematic diagram of the protocol for the purification of PCC from BaCC. FIG. 13B shows microscopic images of BaCC before and after 10% hexane treatment (top panel), and a bar diagram depicting the percentage of cell growth (measured using OD600) of BaCC before and after 10% hexane treatment (bottom panel). FIG. 13C shows an image of an SDS-PAGE gel depicting the starting BaCC material and the final purified PCC product.

FIG. 14 shows a line graph depicting the bioactivity of PCC processed from BaCC with 10% hexane to kill the bacterium and 50% hexane to remove other bacterial contaminants (PCC-Hex/Hex).

FIG. 15 shows a bar graph depicting the bioactivity of PCC processed from BaCC with carvacrol to kill the bacterium (IBaCC), with 50% hexane to remove other bacterial contaminants (PCC-Carv&Hex), and with 10% hexane to kill the bacterium and 50% hexane to remove other bacterial contaminants (PCC-Hex&Hex).

FIG. 16 shows a line graph depicting the bioactivity of PCC processed from BaCC with carvacrol to kill the bacterium and 50% hexane to remove other bacterial contaminants.

### DETAILED DESCRIPTION

Disclosed are compositions of purified nematicidal crystal proteins, methods for making such purified nematicidal crystal proteins, and methods for treating or preventing STH infection by administering to a subject a preparation of purified nematicidal crystal proteins.

### Microbes

In certain embodiments, the bacteria of the disclosure are non-sporulating bacteria. As used herein, the term “non-sporulating bacterium” includes wild-type bacteria that are incapable of producing spores (e.g., certain Gram-negative bacteria) as well as genetic variants of spore-forming bacteria that have been engineered to be defective in sporulation (e.g., certain Gram-positive bacteria). As used herein, unless the context makes clear otherwise, “a mutation resulting in a defect in sporulation” or “a genetic mutation that results in a defect in sporulation” refers to any genetic mutation that results in a defect in a member of the sporulation pathway and/or any genetic mutation that prevents the formation of viable spores. For example, non-sporulating bacteria and their creation are described in International Patent Application No. PCT/US2017/013436, incorporated herein by reference.

In some embodiments, sporulation-deficient bacteria are advantageous. An example of a sporulation-deficient bacterium is a spo0A-Bacillus thuringiensis. Any mutation or combination of mutations that confers sporulation deficiency but that does not substantially affect viability or heterologous gene expression can be used. These mutations include but are not limited to mutations in the following genes: kinA, kinB, spo0A, spo0B, spo0E, spo0F, spo0J, spo0M spoIIB, spoIID, spoIIE, spoIIF, spoIIG, spoIIL, spoIIM, spoIIIA, spoIIIB, spoIIIE, spoIVA, spoIVC, spoIVD, spoVG, spoVK, spoVL, spoVM, spoVN, spoVP, spoVQ, spoVID, σH, σF, σE, σG, and σK. (Silvaggi, J., et al. Unmasking novel sporulation genes in Bacillus subtillus. J Bacteriol. 186, 8089-8095, 2004; Sandman, K., et al. Genetic Analysis of Bacillus subtilis spo Mutations Generated by Tn917-Mediated Insertional Mutagenesis. Genetics. 117, 603-617, 1987; Malvar and Baum, Tn5401 Disruption of the spoOF Gene, Identified by Direct Chromosomal Sequencing, Results in CryIIIIA Overproduction in Bacillus thuringiensis. J Bacteriol. 176, 4750-4753, 1994).

In some embodiments, an engineered sporulation deficiency may also render the host bacterium deficient in production in other crystal proteins and other virulence-associated products of sporulation. For example, spo0A-Bt does not produce Cry5B or other endotoxins such as Cry1, Cry4, or Cry8. In some embodiments, the genes of other Cry proteins and/or accessory proteins may be deleted or inactivated to ensure that no additional Cry proteins are used to form the protein crystal.

In such embodiments of sporulation-deficient bacteria, the sporulation-deficient bacteria may be engineered to express a single crystal protein gene such as Cry5B that is under control of a promoter that is actively and/or highly expressed prior to the sporulation phase of a bacterium, e.g., during the vegetative growth or stationary phase. Such engineered bacteria allow for only a single crystal protein to be expressed, and the resulting nematicidal crystals are homogenously comprised of only a single type of crystal protein. In certain embodiments, the promoter is heterologous (i.e., a non-sporulation specific promoter). In one embodiment, the promoter is a Cry3A, GerA, GNAT, or TadA promoter.

In some embodiments, strains of non-sporulating bacteria that autolyse at the end of their growth cycle may be used. Such autolysing bacteria may be used, such that a homogenization step during the purification process may be avoided or reduced.

Bacteria are particularly applicable to the control of STHs because 1) recombinant bacteria can cheaply express large amounts of Cry proteins prior to administration into the GI tract of a mammalian subject, and Cry proteins so expressed, independent of any Cry proteins that may be secreted by bacteria in the GI tract, have been shown to have a significant impact on STHs, 2) studies using purified Cry protein to treat hookworms, whipworms, and H. bakeri, all in infected rodents, demonstrate that STHs in the mammalian GI tract can ingest and be killed/intoxicated by Cry proteins, 3) recombinant bacteria expressing a therapeutic protein, in which the protein is not purified, are cheaper to produce since no protein purification steps are needed, and 4) recombinant bacteria delivering STH curing proteins (e.g., Cry5B) are more effective that purified proteins (e.g., Cry5B) at the same bio-active protein dose (e.g., total Cry5B) in curing infections.

Microbes of the disclosed compositions and methods include killed and inactivated forms of Bacillus sp., including Bacillus subtilis (e.g., B. subtilis natto, and B. subtilis PY79), B. cereus, (e.g., B. cereus var. Toyoi (Toyocerin), B. cereus var. toyoii), B. toyonensis, B. clausii, B. pumilus and B. thuringiensis. B. subtilis has been extensively characterized as a safely ingested food additive in humans. In certain exemplary embodiments, killed and inactive forms of B. thuringiensis are used.

Other useful bacteria include but are not limited to non-sporulating variants of Lactococcus sp., Bifidobacterium sp., Streptococcus sp., Clostridium sp., Sporolactobacillus sp, Sporosarcina sp., Brevibacillus sp, Leuconostoc sp., Pedicoccus sp., Enterococcus sp. and Escherichia sp. Lactobacillus sp. includes but is not limited to L. lactis, L. casei, L. paracasei, L. acidophilus, L. bulgaricus, L. delbrueckii subsp. bulgaricus, L. helveticus, L. plantarum, L. salivarius, L. reuteri, L. gasseri, and L. animalis. Bifidobacterium sp. includes but is not limited to B. animalis, B. bifidum, B. breve, B. infantis, and B. longum. Streptococcus sp. includes but is not limited to S. thermophilus. Clostridium sp. includes but is not limited to Clostridium butyricum. Sporolactobacillus sp. includes but is not limited to Sporolactobacillus vineae. Sporosarcina sp. includes but is not limited to Sporosarcina pasteurii. Brevibacillus sp. includes but is not limited to Brevibacillus laterosporus.

Still other useful bacteria useful in connection with present disclosure include forms of Gram-negative bacteria. In certain exemplary embodiments, the Gram-negative bacteria include E. coli species (e.g., NISSLE 1917) and Pseudomonas species (e.g., Pseudomonas fluorescens). Exemplary Cry-expressing Gram-negative bacteria which can be killed or inactivated by the methods of the present disclosure include the Cry-expressing E. coli strain of Ge et al. (“Hyperexpression of a Bacillus thuringiensis delta-endotoxin-encoding gene in Escherichia coli: properties of the product”, Gene, 93: 49-54 (1990)) and the P. fluorescens strain of Peng et al. (“A Delta-endotoxin encoded in Pseudomonas fluorescens displays a high degree of insecticidal activity”, App. Microbiol Biotech., (2003), 63:300-306).

### Nematicidal Proteins

As used herein, unless the context makes clear otherwise, “nematicidal protein” refers to any protein that has toxic activity against nematodes or helminthes. Exemplary nematicidal proteins include crystal proteins such as the anthelmintic Cry proteins (e.g., Crickmore et al., 1998 Microbiology and Molecular Biology Reviews 62(3): 807-813; Schnepf et al., 1998 Microbiology and Molecular Biology Reviews 62(3): 775-806; including but not limited to the B. thuringiensis Cry proteins Cry5B (e.g., SEQ ID NO:1) and its subvariants, Cry13A (e.g., SEQ ID NO:2) and its subvariants, Cry14A (e.g., SEQ ID NO:3) and its subvariants, Cry21A (e.g., SEQ ID NOS:4-5) and its subvariants, and Cry6A and its subvariants (e.g., SEQ ID NO:6)) in the bacterium for delivery into a helminth (e.g., roundworm)-infected vertebrate animal gastrointestinal tract via oral dosing (gavage, drinking, eating, pill, capsule, powder, etc.). The Cry proteins are expressed in the cytosol of the bacterium and form crystals, allowing access to the anthelmintic protein after the bacterium lyses or opens up. Nematicidal crystals formed from nematicidal crystal proteins (such as Cry proteins) induce toxicity in worms and helminths by solubilizing or decrystalizing to release the individual nematicidal crystal proteins, thereby allowing the crystal protein to act directly on worms and helminths.

The nematicidal crystals described herein are more stable than the individual crystal proteins of which they are formed, and are resistant to proteolysis. Crystal proteins expressed by bacterium as described herein form crystals of between about 100 kDa and about 170 kDa, between about 110 kDa and about 160 kDa, between about 120 kDa and about 150 kDa, between about 125 kDa and about 145 kDa, or between about 130 kDa and about 140 kDa. These crystal proteins come together to form much larger nematicidal crystals.

Each nematicidal crystal may have a size of about 100 nm, about 200 nm, about 300 nm, about 400 nm, about 500 nm, about 600 nm, about 700 nm, about 800 nm, about 900 nm, about 1000 nm, about 1100 nm, about 1200 nm, about 1300 nm, about 1400 nm, about 1500 nm, about 1600 nm, about 1700 nm, about 1800 nm, about 1900 nm, or about 2000 nm along its longest axis.

In some embodiments, the nematicidal crystals described herein may be between about 100 nm and about 2000 nm, between about 200 and about 2000 nm, between about 300 nm and about 2000 nm, between about 400 nm and about 2000 nm, between about 500 nm and about 2000 nm, between about 600 nm and about 2000 nm, between about 700 nm and about 2000 nm, between about 800 nm and about 2000 nm, between about 900 nm and about 2000 nm, between about 1000 nm and about 2000 nm along its longest axis.

In some embodiments, the nematicidal crystals described herein may be between about 100 nm and about 1000 nm, between about 200 and about 1000 nm, between about 300 nm and about 1000 nm, between about 400 nm and about 1000 nm, between about 500 nm and about 1000 nm, between about 600 nm and about 1000 nm, between about 700 nm and about 1000 nm, between about 800 nm and about 1000 nm, between about 900 nm and about 1000 nm, between about 1000 nm and about 1000 nm along its longest axis.

In certain embodiments, purified nematicidal crystals formed from a first type of nematicidal crystal protein (e.g. Cry5B) may be combined with nematicidal crystals formed from a second type of nematicidal crystal protein (e.g. Cry5C, Cry5D, Cry6A, Cry13A, Cry14A, Cry21A, Cry21B, or Cry55B) in a single pharmaceutical formulation. In such embodiments, a single formulation allows the GI tract to be seeded with multiple forms of purified nematicidal crystals simultaneously. For example, due to the lack of cross-resistance between Cry5B-resistant roundworms and Cry21A-resistant roundworms, simultaneous administration of Cry5B and Cry21A in the gastrointestinal tract may inhibit the development of parasite resistance to the combination therapy.

In the long run, removing antibiotic selection capability (e.g., genetic selection markers) from the plasmids that are used to introduce heterologous Cry protein-encoding sequences, as well as using bacterial strains that are unable to replicate outside the vertebrate host, may be desirable in order to environmentally contain the genetically modified bacteria. For example, LAB (Lactic Acid Bacteria) have been engineered to be autotrophic in thymidine or thymine synthesis such that they can only grow in the vertebrate intestine where thymidine or thymine is present and not in the environment where thymidine or thymine is not present. See, e.g., Steidler L, et al. “Biological containment of genetically modified Lactococcus lactis for intestinal delivery of human interleukin 10.” Nat Biotechnol 21: 785-789 (2003).

Cry-transformed bacteria such as Bacilli or LAB may be cultured and expression of intracellular, membrane-anchored, or secreted Cry protein by such bacteria may be confirmed using antibodies raised against each Cry protein and standard Western blotting or ELISA techniques.

To assess the bioactivity of all constructs, recombinant bacteria expressing Cry protein (full length, truncated, or variants) may be fed to the free-living nematode, C. elegans. Cry protein toxicity on C. elegans using LC50, brood-size, developmental inhibition assays on solid media and in liquid wells may then be quantitated. C. elegans can access the Cry proteins either via protein secreted onto the solid media/into the liquid well or by their ability to grind, open and digest bacteria. Confirmation that the recombinant bacteria are making bioactive Cry proteins may be obtained. Furthermore, the bioactivity (e.g., LC₅₀ in μg/mL) may be quantified and the constructs giving the highest activity determined.

### Truncations, Variants, and Sub-Variants

The crystal proteins may be truncated to enhance their effectiveness. The usefulness of Bt toxins (e.g., crystal proteins) for controlling STHs may be limited by the protein size that STHs can ingest. Some parasitic roundworms poorly ingest proteins larger than about 40 kDa. Thus, the effectiveness of any particular Bt toxin may be limited by size exclusion of proteins that STHs take in and so should be small enough to be readily absorbed by the STH gut while retaining toxic activity. A truncated toxin may be easier to express in bacteria. Producing a truncated toxin also alleviates the requirement that the target STH has the proper proteases present to correctly process full length protoxin (which is inactive) to a truncated, active toxin form. Thus, a truncated toxin is immediately available for intoxication independent of whether the proper protease processing enzymes are present in the STH target. Truncated toxin may also express at a higher level in microbes because truncated toxins are soluble and less likely to form insoluble inclusions in the cell expressing them, which could be toxic to the cell or which could make the toxin fold incorrectly. Accordingly, it is desirable to produce truncated Bt toxin fragments (e.g., crystal protein fragments). Moreover, fragments of certain Bt toxins have been tested and shown to retain toxic activity and have improved biological properties. By “truncated,” when referring to a Bt toxin protein (crystal protein) is meant a Bt toxin protein that is not full-length but retains at least 5%, 10%, 20%, 30%, 40%, 50%, 60%, 70%, 80%, or 90% or more of the toxic activity of a corresponding full-length Bt toxin protein.

“Variants” or “subvariants” of Cry proteins include polypeptides with one or more substitutions, e.g., no more than 20 substitutions, alternatively no more than 10 substitutions, or substitutions at 10% or fewer of the residues, relative to a corresponding wild-type polypeptide or truncated version thereof. The variant, subvariant, or truncated polypeptide has at least 5%, 10%, 20%, 30%, 40%, 50%, 60%, 70%, 80%, or 90% of the activity, e.g., toxic activity, of the corresponding wild-type polypeptide or truncated version. Conservative substitutions include substitutions within the following groups: glycine, alanine, threonine, valine, isoleucine, leucine; aspartic acid, glutamic acid, asparagine, glutamine; serine, cysteine; lysine, arginine; aspartic acid, glutamic acid; serine, threonine; asparagine, glutamine; phenylalanine, tyrosine. One exemplary variant Cry protein is Cry5B with cysteine substituted for serine at position 407 (Ser407Cys) (SEQ ID NO: 7).

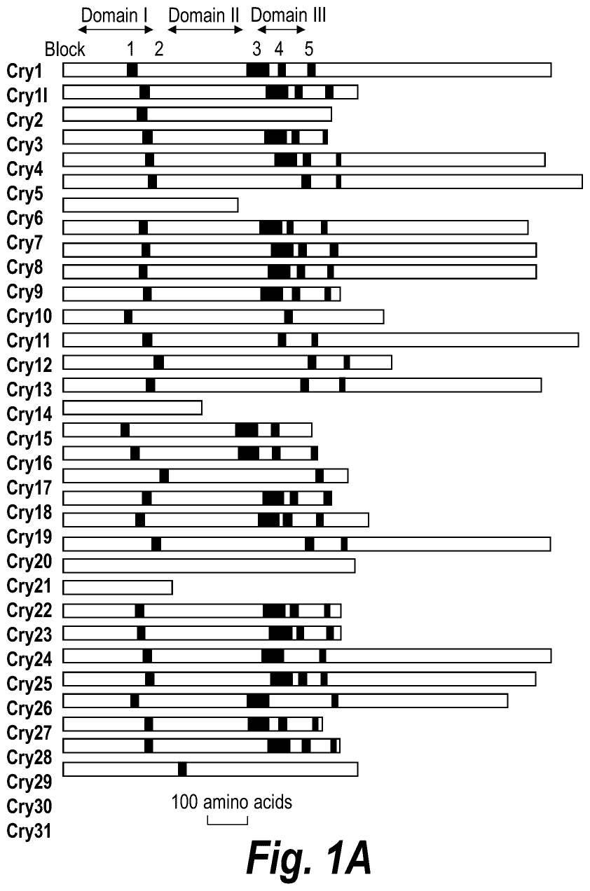

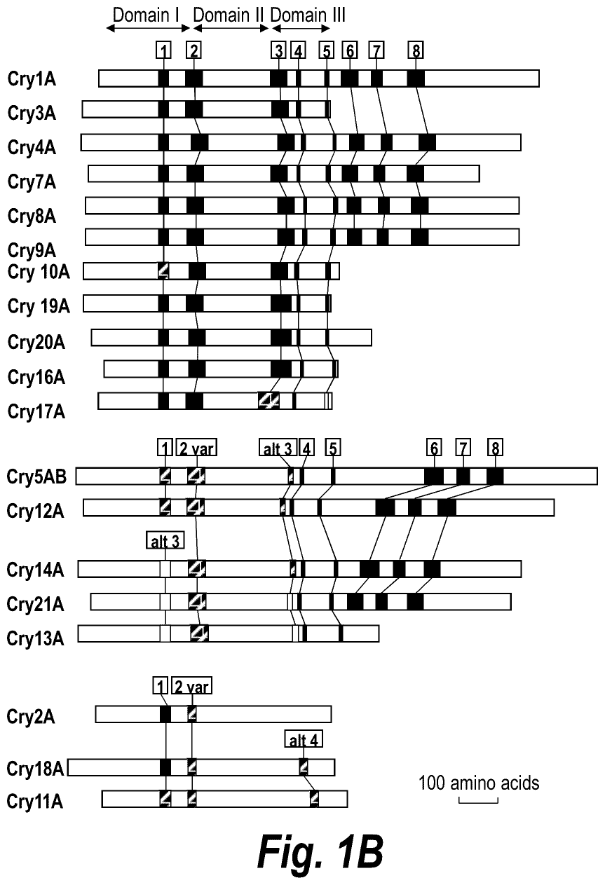

The crystal proteins may be full length, truncated, variants, or subvariants. The truncated crystal protein may include any truncation of the N- and C-termini that still retains toxin activity. The truncated form is not full-length but retains at least 5%, 10%, 20%, 30%, 40%, 50%, 60%, 70%, 80%, or 90% or more of the toxic activity of a corresponding full-length Bt toxin protein. For example, the truncated portion may be truncated between the end of conserved block 5 and the C-terminus of the full-length protein. FIGS. 1A and 1B schematically compare the numbered conserved amino acid blocks (1-5) for a variety of Cry proteins.

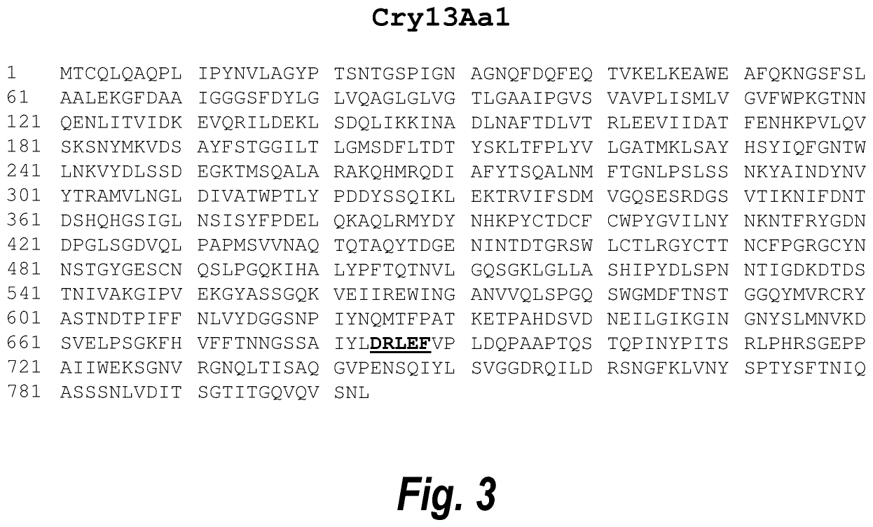

In one embodiment, the truncated crystal protein may contain the toxin domain of the crystal protein and optionally include up to 5, 10, or 20 additional amino acids. The truncated crystal protein may be truncated after a conserved amino acid sequence of block 5 and optionally include up to 5, 10, or 20 additional amino acids. The conserved amino acid sequence of block 5 may contain the motif DRIEF (SEQ ID NO: 23), DRLEF (SEQ ID NO: 24), or some other related sequence as well as surrounding amino acid residues, e.g., three amino acids upstream and two amino acids downstream of this motif. Table 1 shows the block 5 sequences for various Cry proteins. See e.g., Schnepf, E., et al., Bacillus thuringiensis and Its Pesticidal Crystal Proteins, Microbiology and Molecular Biology Reviews 62(3): 775-806, (e.g., at p. 781, FIG. 3) (September 1998); and Crickmore et al., Revision of the Nomenclature for the Bacillus thuringiensis Pesticidal Crystal Proteins, Microbiology and Molecular Biology Reviews 62(3): 807-813 (September 1998). The truncated crystal protein may also be truncated at the N-terminus. For example, the truncated crystal protein may not contain the first about 1, 2, 3, 4, 5, 6, 7, 8, 9, 10, 15, 20, 25, 30, 35, 40, 45, or 50 amino acids at the N-terminus.

Cry protein variants can exhibit at least 50, 55, 60, 65, 70, 75, 80, 85, 86, 87, 88, 89, 90, 91, 92, 93, 94, 95, 96, 97, 98, or 99 percent amino acid sequence identity to a known Cry protein sequence such as any that are disclosed in Crickmore et al., 1998 Microbiology and Molecular Biology Reviews 62(3): 807-813, or in Schnepf et al., 1998 Microbiology and Molecular Biology Reviews 62(3): 775-806, including full length Cry proteins and truncated Cry proteins, Cry protein variants or subvariants thereof. Also contemplated according to certain embodiments are polynucleotides encoding such Cry proteins and truncations and variants thereof.

TABLE 1

| Protein | Block 5 Conserved Group |
|---|---|
| Cry1A | VYIDRIEFVP (SEQ ID NO: 7) |
| Cry3A | VYIDKIEFIP (SEQ ID NO: 8) |
| Cry4A | VLIDKIEFLP (SEQ ID NO: 9) |
| Cry5A | VFLDRIEFIP (SEQ ID NO: 10) |
| Cry5B | LFLDRIEFVP (SEQ ID NO: 11) |
| Cry7A | FYVDSIEFIP (SEQ ID NO: 12) |
| Cry8A | VYIDRIEFIP (SEQ ID NO: 13) |
| Cry9A | VYVDRIEFIP (SEQ ID NO: 14) |
| Cry10A | IYIDKIEFIP (SEQ ID NO: 15) |
| Cry12A | MVLDRIEFVP (SEQ ID NO: 16) |
| Cry13A | IYLDRLEFVP (SEQ ID NO: 17) |
| Cry14A | IFIDRIEFIP (SEQ ID NO: 18) |
| Cry19A | LILDKIEFLP (SEQ ID NO: 19) |
| Cry20A | FVLDKIELIP (SEQ ID NO: 20) |
| Cry21A | LFLDRIEFIS (SEQ ID NO: 21) |
| Consensus | i-iDkIEFiP (SEQ ID NO: 22) |

In Table 1, the consensus sequence denotes the positions at which at least 75% of the aligned proteins in the group have an identical or conserved amino acid sequence. An uppercase letter in the sequence indicates that at least 75% of the residues at that position are identical. A lowercase letter indicates that at least 75% of the residues at that position are conserved. Conserved amino acids fall into the following groups: a (A, G, S, T, or P); d (D, E, N, or Q); f (F, W, or Y), I (I, L, M, or V), and k (K or R).

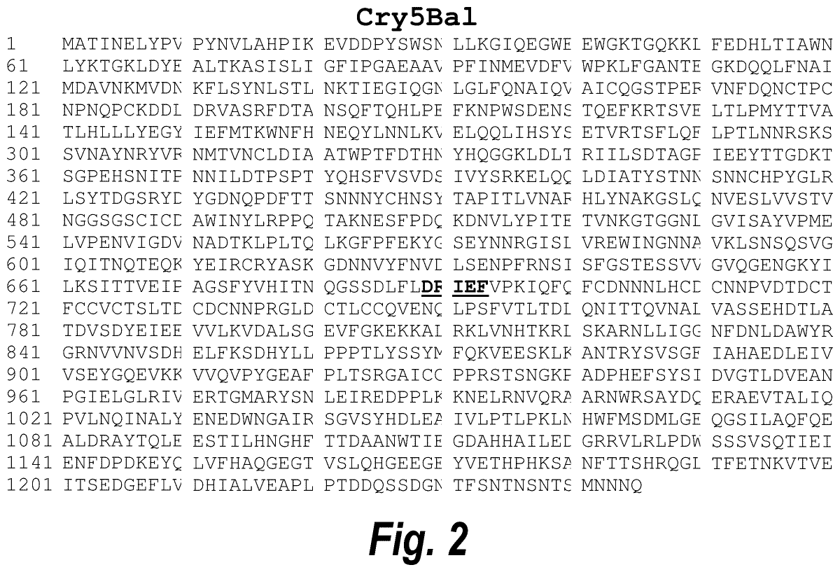

The truncated crystal protein may be a truncated form of Cry5B such as B. thuringiensis Cry5B (FIG. 2). Truncated Cry5B may extend from about amino acid 1, 2, 3, 4, 5, 6, 7, 8, 9, 10, 15, 20, 25, 30, 35, 40, 45, or 50 to at least about amino acid 693. The truncated form of Cry5B may optionally include up to an additional 5, 10, 20, 30, 40, or 50 amino acids from the C-terminus after conserved block 5, e.g., through about 698, 703, 713, 723, 733, or 743.

The truncated crystal protein may be a truncated form of Cry13A such as B. thuringiensis Cry13A (FIG. 3). Truncated Cry13A may extend from about amino acid 1, 2, 3, 4, 5, 6, 7, 8, 9, 10, 15, 20, 25, 30, 35, 40, 45, or 50 to at least about amino acid 688. The truncated form of Cry13A may optionally include up to an additional 5, 10, 20, 30, 40, or 50 amino acids from the C-terminus after conserved block 5, e.g., through about 693, 698, 708, 718, 728, or 738.

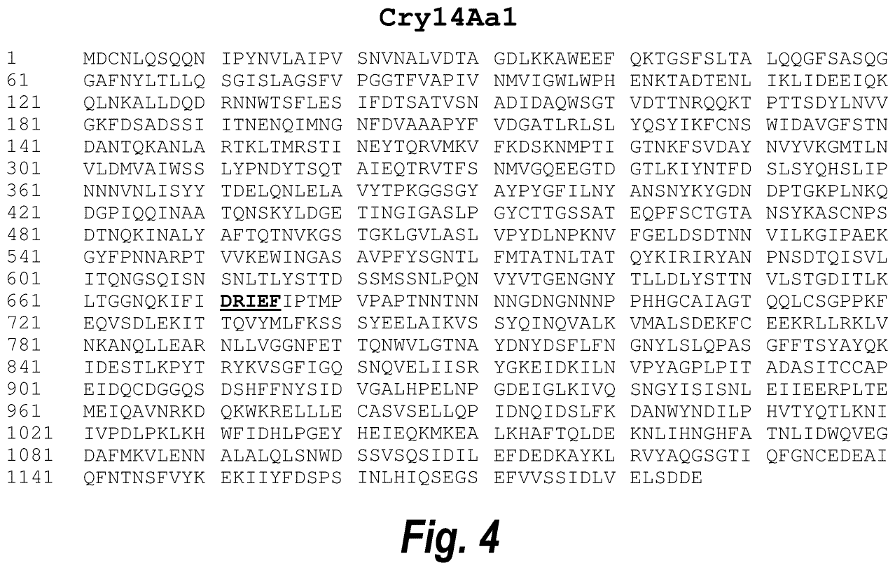

The truncated crystal protein may be a truncated form of B. thuringiensis Cry14A (FIG. 4). Truncated Cry14A may extend from about amino acid 1, 2, 3, 4, 5, 6, 7, 8, 9, 10, 15, 20, 25, 30, 35, 40, 45, or 50 to at least about amino acid 675. The truncated form of Cry14A may optionally include up to an additional 5, 10, 20, 30, 40, or 50 amino acids from the C-terminus after conserved block 5, e.g., through about 680, 685, 695, 705, 715, or 725.

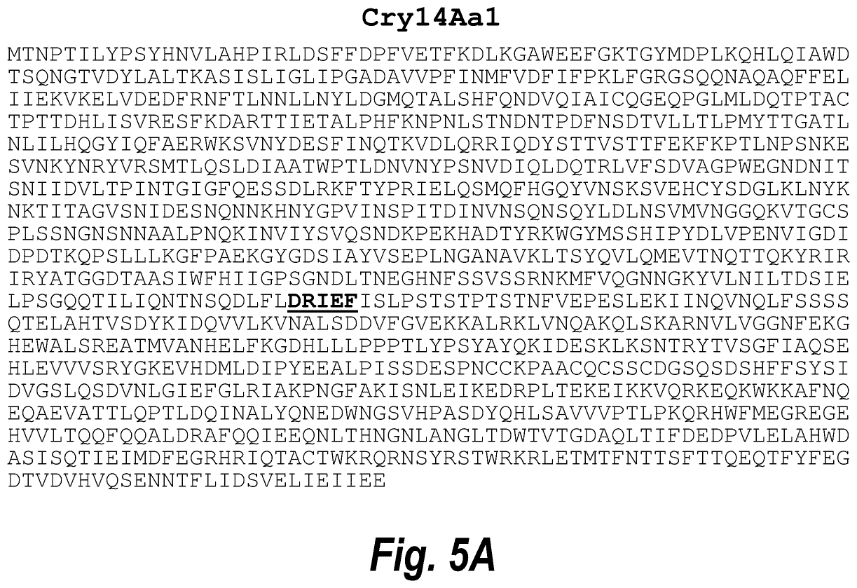

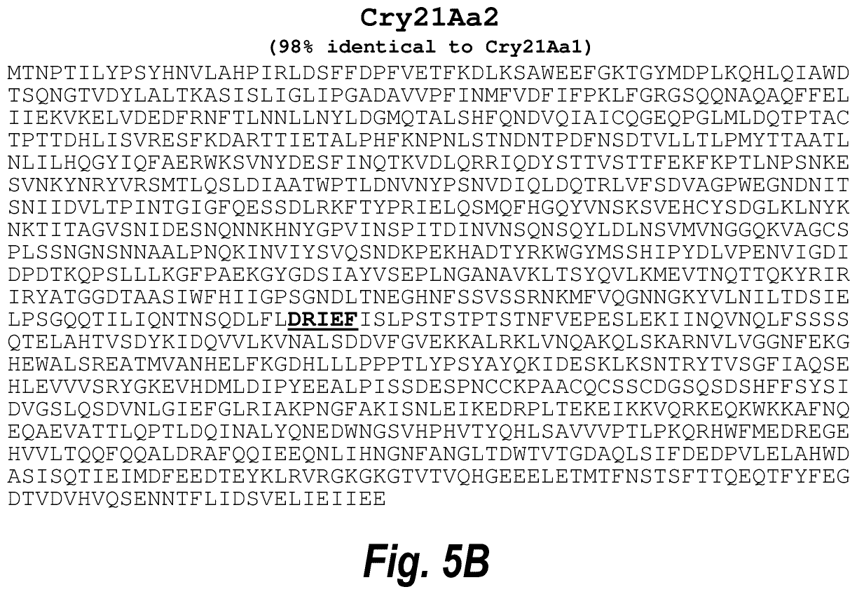

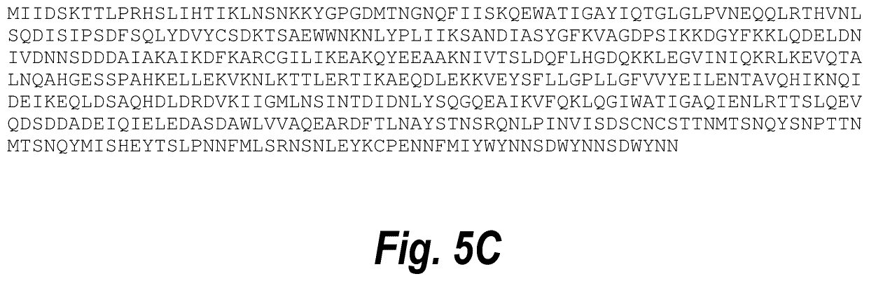

The truncated crystal protein may be a truncated form of Cry21A such as B. thuringiensis Cry21Aa1 (FIG. 5A) or Cry21Aa2 (FIG. 5B). Truncated Cry21A may extend from about amino acid 1, 2, 3, 4, 5, 6, 7, 8, 9, 10, 15, 20, 25, 30, 35, 40, 45, or 50 to at least about amino acid 685. The truncated form of Cry21A may optionally include up to an additional 5, 10, 20, 30, 40, or 50 amino acids from the C-terminus after conserved block 5, e.g., through about 690, 695, 705, 715, 725, or 735.

Nucleic acid molecules encoding amino acid sequence variants, truncated versions, or both, of a Cry protein are prepared by a variety of methods known in the art. These methods include, but are not limited to, isolation from a natural source (in the case of naturally occurring amino acid sequence variants) or preparation by, for example, oligonucleotide-mediated (or site-directed) mutagenesis, PCR mutagenesis, and cassette mutagenesis of an earlier prepared variant or a non-variant version of protein. Moreover, the present technology includes synthetic nucleic acid molecules where nucleotides are modified to include codons preferred in a particular organism, remove codons rarely used in a particular organism, or remove sequences that may inhibit transcription or RNA processing and the like.

Cry protein truncations may at least include conserved blocks 1-5. As seen in FIGS. 1A and 1B, alignment of known Cry toxins reveals five conserved sequence blocks (blocks 1-5) that are common to a majority of the proteins and are thought to be located in the active toxin domain. See de Maagd, R. A., et al. “How Bacillus thuringiensis has evolved specific toxins to colonize the insect world.” Trends in Genetics 17(4): 193-99 (April 2001). Comparison of the carboxy-terminal halves of the sequences have suggested the presence of three additional blocks that lie outside of the active toxic core. See Schnepf, E., et al. “Bacillus thuringiensis and Its Pesticidal Crystal Proteins.” Microbiology and Molecular Biology Reviews 62(3): 775-806 (September 1998). Thus, Cry protein truncations may be truncated after the conserved amino acid sequence of block 5 (e.g., DRIEF (SEQ ID NO: 23) or DRLEF (SEQ ID NO: 24)). Alternatively, Cry protein truncations may be truncated after the conserved amino acid sequence of block 5 (e.g., DRIEF (SEQ ID NO: 23) or DRLEF (SEQ ID NO: 24)) plus an additional about 2, 3, 4, 5, 10, 15, 20, 25, 30, 35, 40, 45, or 50 amino acids of the c-terminal domain.

The complete amino acid sequence of Cry5Ba1 is listed in FIG. 2. The conserved amino acid sequence DRIEF (SEQ ID NO: 23) in Cry5B ends at amino acid number 693. Thus, a truncated form of Cry5B may include at least amino acids 50 through about 693. A truncated form of Cry5B may extend from about amino acid 1, 2, 3, 4, 5, 6, 7, 8, 9, 10, 15, 20, 25, 30, 35, 40, 45, or 50 to at least about amino acid 693. Alternatively, or in addition to, a truncated form of Cry5B may include about 5, 10, 15, 20, 25, 30, 35, or 40 additional amino acids of the c-terminal domain.

The complete amino acid sequence of Cry13Aa1 is listed in FIG. 3. The conserved amino acid sequence DRLEF (SEQ ID NO: 24) in Cry13A ends at amino acid number 688. Thus, a truncated form of Cry13A may include at least amino acids 50 through about 688. A truncated form of Cry5B may extend from about amino acid 1, 2, 3, 4, 5, 6, 7, 8, 9, 10, 15, 20, 25, 30, 35, 40, 45, or 50 to at least about amino acid 688. Alternatively, or in addition to, a truncated form of Cry13A may include about 5, 10, 15, 20, 25, 30, 35, or 40 additional amino acids of the c-terminal domain.

The complete amino acid sequence of Cry14Aa1 is listed in FIG. 4. The conserved amino acid sequence DRIEF (SEQ ID NO: 23) in Cry14A ends at amino acid number 675. Thus, a truncated form of Cry14A may include at least amino acids 50 through about 675. A truncated form of Cry5B may extend from about amino acid 1, 2, 3, 4, 5, 6, 7, 8, 9, 10, 15, 20, 25, 30, 35, 40, 45, or 50 to at least about amino acid 675. Alternatively, or in addition to, a truncated form of Cry14A may include about 5, 10, 15, 20, 25, 30, 35, or 40 additional amino acids of the c-terminal domain.

The complete amino acid sequence of Cry21Aa1 and Cry21Aa2 are listed in FIG. 5A and FIG. 5B, respectively. The amino acid sequence of Cry21Aa2 is about 98% identical to the sequence of Cry21Aa1. The conserved amino acid sequence DRIEF (SEQ ID NO: 23) in Cry21A ends at amino acid number 685. Thus, a truncated form of Cry21A may include at least amino acids 50 through about 685. A truncated form of Cry5B may extend from about amino acid 1, 2, 3, 4, 5, 6, 7, 8, 9, 10, 15, 20, 25, 30, 35, 40, 45, or 50 to at least about amino acid 685. Alternatively, or in addition to, a truncated form of Cry21A may include about 5, 10, 15, 20, 25, 30, 35, or 40 additional amino acids of the c-terminal domain.

### Anthelmintic Experiments

Once heterologous Cry protein expression and bioactivity are confirmed in a desired bacterium, the modified bacteria may be used for curative-type and preventative-type anthelmintic experiments.

Antibody production: Antibodies against recombinant Cry proteins (e.g., Cry5B, Cry21A, Cry14A, Cry13A, and Cry6A, full length and truncated proteins) may be produced and purified according to standard methodologies (e.g., Current Protocols in Immunology, John Wiley & Sons, New York, N.Y. (2009).

Bioactivity tests: To assess the bioactivity of all constructs, recombinant bacilli or other bacteria expressing heterologous Cry proteins are fed to the free-living nematode, C. elegans. C. elegans can access the Cry proteins either via protein secreted onto the solid media/into the liquid well, by protein naturally released as bacteria break open, or by their ability to grind and digest bacteria to open the bacterial cells.

Rodent and parasite tests: Three intestinal parasitic nematodes—H. bakeri (small intestine nematode parasite) in mice, and Trichuris muris (whipworm) in mice, and A. ceylanicum (hookworm) in hamsters are tested. The tests address: 1) where in the GI tract do heterologous Cry-expressing bacteria reside and for how long; and 2) how do these bacteria affect the acquisition and progression of intestinal nematode parasites.

Parasite tests: Naïve (uninfected) mice are gavaged with the best heterologous Cry-protein expressing recombinant bacterial strain(s) based on expression and bioactivity. Protect against progression test: Mice are infected with H. bakeri. Two weeks later, infected mice are treated with heterologous Cry-protein expressing or control bacteria, respectively. Intestinal worm burdens and fecal egg counts are used to determine if the recombinant bacteria provide anthelmintic therapy in mice with pre-existing nematode infections.

### Exemplary Parasites

The disclosed methods relate to the control of parasitic worms, e.g., nematodes and platyhelminths, using crystal proteins from Bacillus and their derivatives. Parasitic worms within the scope of the invention include but are not limited to those in Class Adenophorea, e.g., Order Mononchida, Family Plectidae, and Order Stichosomida, Family Mermithidae and Tetradonematidae; Class Secernentea, e.g., Order Rhabditida, Family Carabonematidae, Cephalobidae, Chambersiellidae, Heterorhabditidae, Oxyuridae, Panagrolaimidae, Rhabditidae, Steinernematidae, Syrphonematidae, Syrphonematidae, or Thelastomatidae; Order Spirurida, Family Filariidae, Onchocercidae, Physalopteridae, Syngamidae, Spiruridae, Subuluridae, or Thelaziidae; Order Diplogasterida, Family Diplogasteridae; and Order Tylenchida, Family Allantonematidae, Aphelenchidae, Aphelenchoididae, Entaphelenchidae, Fergusobiidae, Phaenopsitylenchidae, Sphaerulariidae, Anguinidae, Dolichodoridae, Belonolaimidae, Pratylenchidae, Hoplolamidae, Heteroderidae, Criconematidae, Tylenchulidae or Tylenehidae. In one embodiment, the parasite is from Class Secernentea, Order Ascaridida, Family Ascarididae; Class Adenophorea, Order Trichurida, Family Trichuridae; Class Secernentea, Order Strongylida, Family Ancylostomatidae (ancylostomidae) or Trichostrongylidae; or Class Secernentea, Order Spirurida, Family Dracunculidae, Filariidae, or Onchocercidae.

The parasite may be a helminth. Helminths within the scope of the invention include but are not limited to those from Phylum Annelida, Class Polychaetae, Class Myzostomida, Class Clitellata, Subclass Hirudinea, Order Gnathobdellidae, Order Rhynchobdellidae; Phylum Platyhelminthes (Flatworms), Class Turbellaria, Class Monogenea, Order Monopisthocotylea, Order Polyopisthocotylea, Class Trematoda, Subclass Aspidogasrea, Subclass Digenea; Super Order Anepitheliocystida, Order Strigeatida, Family Schistosomatidae, Subfamily Schistosomatinae, Genus Schistosoma, Order Echinostomatida, Family Fasciolidae, Family Paramphistomatidae, Family Echinostomatidae; Super Order Epitheliocystida, Order Plagiorchiida, Family Dicrocoeliidae, Family Troglotrematidae, Order Opisthorchiida, Family Heterophyidae, Family Opisthorchiidae, Class Cestoda, Subclass Cestodaria, Subclass Eucestoda, Order Pseudophyllidea, Family Diphyllobothriidae, Order Cyclophyllidea, Family Taeniidae, Family Hymenolepididae, Family Dilepididae, Family Mesocestoididae, Order Tetraphyllidea, Order Proteocephalata, or Order Spatheobothridea. For example, Cry proteins with the scope of the invention may be employed to prevent, inhibit or treat Roundworm, Whipworm, Hookworm, Schistosome, or Trematodes.

The parasite may also be gastrointestinal tract parasitic roundworms/nematodes. The gastrointestinal tract parasitic roundworms/nematodes may include but are not limited to the following species: Haemonochus, Cooperia, Ostertagia, Trichostrongylus, Teladorsagia, Nematodirus, Ancylostoma, Cyathostominea/Cyathostomin/Cyathostome, Strongylus, Parascaris, Ascaris, Trichuris, Oesophagostomum/Oesophagustomum, Trichiuris, Bunostomum, Oxyuris, Chabertia, Habronema, Draschia, Triodontophorus, Toxocara, Toxascaris, and Uncinaria. Haemonochus species includes but is not limited to Haemonchus contortus and Haemonchus placei, Cooperia species includes but is not limited to Cooperia oncophora, Cooperia pectinata, and Cooperia curticei. Ostertagia species includes but is not limited to Ostertagia ostertagi, Ostertagia (Teladorsagia) circumcincta, and Ostertagia trifurcate. Trichostrongylus species includes but is not limited to Trichostrongylus axei, Trichostrongylus colubriformis, and T. circumcincta. Teladorsagia species includes but is not limited to Teladorsagia (Ostertagia) circumcincta. Nematodirus species includes but is not limited to Nematodirus spathiger. Ancylostoma species includes but is not limited to Ancylostoma caninum, Ancylostoma braziliense, and Ancylostoma tubaeforme.

Cyathostominea/Cyathostomin/Cyathostome nematodes are also included. Strongylus species (small and large) includes but is not limited to Strongylus vulgaris, Strongylus equinus, and Strongylus edentatus. Parascaris species includes but is not limited to Parascaris equorum. Strongyloides species includes but is not limited to Strongyloides westeri. Ascaris species includes but is not limited to Ascaris suum. Trichuris species includes but is not limited to Trichuris globulosa, Trichuris suis, Trichuris campanula, and Trichuris vulpis. Oesophagostomum/Oesophagustomum species includes but is not limited to Oesophagustomum dentatum, Oesophagustomum quadrispinulatum, Oesophagostomum columbianum, and Oesophagostomum venulosum. Trichiuris species includes but is not limited to Trichiuris ovis. Bunostomum species includes but is not limited to Bunostomum trigonocephalum. Oxyuris species includes but is not limited to Oxyuris equi (pin worms). Chabertia species includes but is not limited to Chabertia ovina. Habronema species includes but is not limited to Habronema microstoma and Habronema muscae. Draschia species includes but is not limited to Draschia megastoma. Triodontophorus species includes but is not limited to Triodontophorus minor and Triodontophorus serrates. Toxocara species includes but is not limited to Toxocara canis and Toxocara cati. Toxascaris species includes but is not limited to Toxascaris leonine. Uncinaria species includes but is not limited to Uncinaria stenocephala. Human parasitic roundworms of the gastrointestinal tract include but are not limited to the hookworms Ancylostoma duodenale and Necator americanus, the whipworm Trichuris trichiura, the roundworm Ascaris lumbricoides, the threadworm Strongyloides stercoralis, and the pinworm Enterobius vermiculari.

### Additional Therapeutic Agents

In certain embodiments, the pharmaceutical compositions of the invention are administered in combination with at least one additional therapeutic agent. This additional agent can be, for example, a small molecule or a polypeptide (including antibodies and fragments thereof). In a further embodiment, the additional therapeutic is a nicotinic acetylcholine receptor agonist. In certain embodiments, the additional therapeutic agent is administered simultaneously with the pharmaceutical compositions of the invention. In certain embodiments, the additional therapeutic agent is administered sequentially (and in either order) with the pharmaceutical composition. In certain embodiments, the nicotinic acetylcholine receptor agonist is from the levamisole family of nicotinic acetylcholine receptor agonists. In certain embodiments, the nicotinic acetylcholine receptor agonist is levamisole. In certain embodiments, the levamisole is administered in an amount of about 0.1 mg/kg to about 5.0 mg/kg. In certain embodiments, the nicotinic acetylcholine receptor agonist is pyrantel or tribendimidine. In certain embodiments, the pyrantel is administered in an amount of about 1.0 mg/kg to about 15.0 mg/kg. In certain embodiments, the tribendimidine is administered in an amount of about 0.25 mg/kg to about 10 mg/kg.

### Administration, Dosage Forms, Pharmaceutical Compositions

The present invention contemplates administration of purified crystal proteins to the gastrointestinal tract of a subject. The pharmaceutical compositions may thus be formulated for oral administration. Oral administration is preferably in an aqueous suspension, emulsion, powder or solid. The composition may be formulated into a food or added to food by the user prior to consumption. Administration to the gastrointestinal tract may also be in the form of an anal suppository (e.g., in a gel or semi-solid formulation). All such formulations are made using standard methodologies.

The methods of treatment disclosed herein are typically practiced on any animal where inhibiting pathogen or parasites is desired. In certain embodiments, the animal is a human. However, the animal can be any livestock or zoological specimen where such inhibition of parasites/pathogens provides economic and health benefits. Any animal can benefit by the claimed methods, including birds, reptiles, mammals such as horses, cows, sheep, goats, pigs, and the like domesticated animals, or any of a variety of animals of zoological interest. Other purposes are readily apparent to one skilled in the arts of nutrient absorption, feed utilization and bioavailability.

The present invention further contemplates a therapeutic system for treating, reducing and/or controlling parasitic infections. Typically, the system is in the form of a package containing a therapeutic composition of the present invention, or in combination with packaging material. The packaging material includes a label or instructions for use of the components of the package. The instructions indicate the contemplated use of the packaged component as described herein for the methods or compositions of the invention. By way of example, and not of limitation, a system can comprise one or more unit dosages of a therapeutic composition according to the present invention. Alternatively, the system can alternately contain bulk quantities of a therapeutic composition. The label contains instructions for using the therapeutic composition in either unit dose or in bulk forms as appropriate, and may also include information regarding storage of the composition, disease indications, dosages, routes and modes of administration and the like information.

Furthermore, depending upon the particular contemplated use, the system may optionally contain either combined or in separate packages one or more of the following components: bifidogenic oligosaccharides, flavorings, carriers, and the like components. One embodiment comprises unit dose packages of purified nematicidal crystal protein for use in combination with a conventional liquid product, together with instructions for combining the purified nematicidal crystal protein with the formula for use in a therapeutic method.

Different dosage regimens may be used in the disclosed methods. In some embodiments, a daily dosage is administered once, twice, three times, or four times a day for one, two, three, four, five, six, seven, eight, nine, or ten days. In some embodiments, a once- or twice-daily dosage is administered every other day.

Administration of the compositions containing the active ingredients effective in inhibiting parasite growth in the intestine and in feces generally consist of one to ten unit dosages of 10 mg to 10 g per dosage of the composition for one day up to one month for a human of approximately 100 kg body weight. Unit dosages are generally given once every twelve hours and up to once every four hours. Preferably two to four dosages of the composition per day, each comprising about 0.1 g to 50 g per dosage, for one to seven days are sufficient to achieve the desired result.

In various specific embodiments, an effective dose of a composition of the present disclosure can be in a range of from about 0.01 mg/kg to about 100 mg/kg for an adult patient, more preferably between about 0.1 mg/kg and about 10 mg/kg of the disclosed composition. Effective doses can be administered to a subject at any suitable frequency, e.g., at least once a week, preferably once a day. Pediatric dosages may be in the range of 15% to 90% of adult dosages.

In other embodiments, a constant dosage of the composition can be administered over time, for example about 2 gm to about 4 gm per day, up to about 6 g to about 10 g per day, depending on the severity of the physiological condition. Once the infection has been effectively ameliorated, the subject can in many instances decrease the dosage to about 2 gm to about 4 gm per day for maintenance purposes. The desired dose may be presented in multiple (e.g., two, three, four, five, six, or more) sub-doses administered at appropriate intervals throughout the day.

The pharmaceutical compositions of the invention can be administered via any of the accepted modes of administration or agents known in the art. However, oral administration is preferred because this route of delivery delivers the purified nematicidal crystal protein to the GI tract. The dosage form can be, for example, a solid, semi-solid, lyophilized powder, or liquid dosage forms, such as for example, tablets, pills, soft elastic or hard gelatin capsules, powders, solutions, suspensions, suppositories, aerosols, or the like, and can be in unit dosage forms suitable for simple administration of precise dosages. One exemplary embodiment of the dose form is a capsule containing the composition of the disclosure including the purified nematicidal crystal protein in a dried form, blended with pharmaceutical carrier. The capsule for such a dose form can be of any suitable type, e.g., a gelatin capsule of a conventional variety.

The physiologically compatible carrier medium with which the purified nematicidal crystal proteins are employed, can be of any simple type, e.g., a pharmaceutically acceptable carrier such as fructo-oligo-saccharide (FOS) medium, or other soluble fiber, sugar, nutrient or base material for the composition, with which the bacterial species can be formulated, e.g., in an orally administrable form. Other carrier media include mannitol, inulin (a polysaccharide), polydextrose, arabinogalactan, polyols lactulose, lactitol, etc. A wide variety of materials can be used as carrier material in the practice of the present disclosure, as will be apparent to those of ordinary skill in the art, based on the description herein.

The carrier medium, when present, can be blended with the bacterial species in any suitable amounts, such as an amount of from 5% to 95% by weight of carrier medium, based on the total weight of the bacterial species and the carrier medium, in various embodiments. In other embodiments, the amount of carrier medium may be in a range having a lower limit of any of 5%, 10%, 12%, 15%, 20%, 25%, 28%, 30%, 40%, 50%, 60%, 70% or 75%, and an upper limit, higher than the lower limit, of any of 20%, 22%, 25%, 28%, 30%, 40%, 50%, 60%, 70%, 75%, 80%, 85%, 90%, and 95%. The amount of carrier medium in a specific embodiment may be determined based on considerations of the specific dose form, relative amounts of the purified nematicidal crystal protein, the total weight of the composition including the carrier medium and the bacterial species, and the physical and chemical properties of the carrier medium, and other factors, as known to those of ordinary skill in the probiotic and pharmaceutical formulation art.

In certain embodiments, the purified nematicidal crystal proteins are formulated in a composition that protects the Cry proteins from the acid environment of the stomach. Accordingly, the invention includes a composition containing a purified nematicidal crystal protein and a pharmaceutically-acceptable acid-resistant (“enteric”) carrier. By acid-resistant is meant that the carrier or coating does not dissolve in an acidic environment. An acidic environment is characterized by a pH of less than 7. The acid-resistant carrier is resistant to acids at pH less than about 4.0. Preferably, the carrier does not dissolve in pH 2-3. Most preferably, it does not dissolve in pH of less than 2. To purified nematicidal crystal proteins from stomach acids, the purified nematicidal crystal protein are coated or encapsulated with the acid-resistant carrier.

In certain embodiments, the coating is pH-sensitive. For example, the coating may dissolve after the pH is greater than 4.0. For example, the coating dissolves in a neutral environment as is encountered in the small intestine, and does not dissolve in an acidic environment as is encountered in the stomach. Alternatively, the enteric coating dissolves when exposed to specific metabolic event such as an encounter with a digestive enzyme that is found in the small intestine. For example, the coating is digested by a pancreatic enzyme such as trypsin, chymotrypsin, or a pancreatic lipase. The formulation is hydrated in the small intestine. Digestion or dissolution of the coating allows liberation of purified nematicidal crystal proteins, e.g., purified Cry5B, into the intestine.

In other embodiments, purified nematicidal crystal proteins are stabilized in a gel or paste such as an anhydrous carbohydrate paste. In alternate formulations, the purified nematicidal crystal proteins are lyophilized and/or suspended in a gel or paste. Enteric coating materials are known in the art, e.g., malic acid-propane 1,2-diol. Cellulose derivatives, e.g., cellulose acetate phthalate or hydroxypropyl methylcellulose phthalate (HPMCP), are also useful in enteric acid-resistant coatings. Other suitable enteric coatings include cellulose acetate phthalate, polyvinyl acetate phthalate, hydroxypropylmethylcellulose phthalate and anionic polymers of methacrylic acid and methyl methacrylate. Another suitable enteric coating is a water emulsion of ethylacrylate methylacrylic acid copolymer, or hydroxypropyl methyl cellulose acetate succinate (HPMAS). (See, e.g., U.S. Pat. No. 5,591,433). An enteric coating is designed to resist solution in the stomach and to dissolve in the neutral or alkaline intestinal fluid.

In certain embodiments, the purified nematicidal crystal proteins are preferably formed into dry powders. Suitable drying methods include a natural drying, a forced-air drying, a spray drying, a freeze drying, and the like. Of those, a spray drying, drum drying or a forced-air drying are preferably used. A protective agent such as skim milk, sodium glutamate, and saccharides may be used in a time of drying. Saccharides, glucose, and trehalose may be used. In an example of freeze-drying, purified crystal protein may be frozen at −80° C. and then put into a FreeZone 1 Liter Benchtop Freeze Dry System (Labconco catalog number 7740020). The condenser is set to −60° C. and the vacuum is set at 22 mTor. The samples are freeze-dried overnight. In an example of spray-drying, a PCC sample at 10% solids w/v is spray dried using a Yamato Pulvis GB22 (or any other spray drying system) through a 100 micron atomizer nozzle at 5 mLs/min with atomizing air set at 1 Kgf/m², drying air set at 0.21 m³/min, inlet/outlet temperatures set at 98° C./59° C., respectively.

Auxiliary and adjuvant agents may include, for example, preserving, wetting, suspending, sweetening, flavoring, perfuming, emulsifying, and dispensing agents. Prevention of the action of contaminating microorganisms, if desired, can be accomplished using various antibacterial and antifungal agents, such as, parabens, chlorobutanol, phenol, sorbic acid, and the like. Isotonic agents, such as sugars, sodium chloride, and the like, may also be included.

Solid dosage forms can be prepared with coatings and shells, such as enteric coatings and others well-known in the art. They can contain pacifying agents and can be of such composition that they release the active compound or compounds in a certain part of the intestinal tract in a delayed manner. Examples of embedded compositions that can be used are polymeric substances and waxes. The active compounds also can be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, etc., the active agent (such as the purified nematicidal crystal protein), and optional pharmaceutical adjuvants in a carrier, such as, for example, water, saline, aqueous dextrose, glycerol, ethanol and the like; solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethyl formamide; oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan; or mixtures of these substances, and the like, to thereby form a solution or suspension.

Actual methods of preparing such dosage forms are known, or will be apparent, to those skilled in this art. Reference is made, for example, to Remington's Pharmaceutical Sciences, 18th ed. (Mack Publishing Company, Easton, Pa., 1990).

### Methods

The methods are directed to treating a parasitic worm or helminth infection in a subject comprising administering to the subject a therapeutically effective amount of a composition comprising isolated native, bioactive nematicidal crystals formed from a single type of nematicidal crystal protein. The pharmaceutical composition is substantially free of any bacterial spores or host bacterial proteins other than nematicidal crystal protein in the form of a crystal.

The methods are also directed to reducing the severity of a parasitic worm or helminth infection comprising administering to the subject a therapeutically effective amount of a pharmaceutical composition comprising isolated native, bioactive nematicidal crystals formed from a single type of nematicidal crystal protein, wherein the pharmaceutical composition is substantially free of any bacterial spores or host bacterial proteins other than nematicidal crystal protein in the form of a crystal.

### Selected Definitions

As used herein, unless the context makes clear otherwise, “treatment,” and similar words such as “treated,” “treating” etc., indicates an approach for obtaining beneficial or desired results, including and preferably clinically desirable results. Treatment can involve optionally either the amelioration of symptoms of the disease or condition, or the delaying of the progression of the disease or condition.

As used herein, unless the context makes clear otherwise, “subject” means a vertebrate, such as a mammal. The mammal can be a feline, a rodent, a canine, a bovine, an equine, a swine, a caprine, an ovine, or a primate. In some embodiments, the subject is a human.

As used herein, unless the context makes clear otherwise, “reducing the likelihood of occurrence,” “prevention,” and similar words such as “prevented,” “preventing” etc., include approaches for preventing, inhibiting, or decreasing the likelihood of the onset or recurrence of a disease or condition, in a manner that exhibits statistical significance, for example, when compared to the results obtained when the indicated method steps are omitted. Similarly, also included are preventing, inhibiting, or decreasing the likelihood of the occurrence or recurrence of the symptoms of a disease or condition, or optionally delaying the onset or recurrence of a disease or condition, or delaying the occurrence or recurrence of the symptoms of a disease or condition. As used herein, “prevention” and similar words also include reducing the intensity, effect, symptoms and/or burden of a disease or condition prior to onset or recurrence of the disease or condition. Methods according to these and related embodiments may be practiced using an effective amount or a therapeutically effective amount of an agent that substantially eradicates, reduces the severity of, or reduces the likelihood of occurrence of a soil-transmitted helminth (STH) infection. As used herein, an “effective amount” or a “therapeutically effective amount” of a composition, agent or substance is that amount sufficient to obtain a desired biological effect, such as beneficial results, including clinical results.

As used herein, unless the context makes clear otherwise, “food grade oil” includes oils suitable for ingestion by humans or animals. Exemplary food grade oils include, but are not limited to, oils extracted from vegetables, such as corn oil, soybean oil, coconut oil, cottonseed oil, olive oil, palm oil, peanut oil, rapeseed or canola oil, safflower oil, sunflower oil. Food grade oil includes, but is not limited to, nut oils, e.g. almond oil, beech nut oil, brazil nut oil, cashew oil, hazelnut oil, macadamia nut oil, mongongo nut oil, pecan oil, pine nut oil, pistachio oil, walnut oil, and pumpkin seed oil. Food grade oils include oils from citrus, fruit, melons and gourd seeds, or any edible plant.

In certain preferred embodiments, the compositions described herein for treating or reducing the severity or likelihood of occurrence of an STH infection are formulated as pharmaceutical compositions, which will preferably be formulated for oral delivery. Pharmaceutical compositions are formulated so as to allow the agent(s) contained therein to be bioavailable upon administration of the composition to a human.

It will be appreciated that the practice of the several embodiments of the present invention will use, unless indicated specifically to the contrary, conventional methods in virology, immunology, microbiology, molecular biology and recombinant DNA techniques that are within the skill of the art, and many of which are described below for the purpose of illustration. Such techniques are explained fully in the literature. See, e.g., Current Protocols in Molecular Biology or Current Protocols in Immunology, John Wiley & Sons, New York, N.Y. (2009); Ausubel et al., Short Protocols in Molecular Biology, 3^{rd} ed., Wiley & Sons, 1995; Sambrook and Russell, Molecular Cloning: A Laboratory Manual (3rd Edition, 2001); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984) and other like references.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well-known and commonly used in the art. Standard techniques may be used for recombinant technology, molecular biological, microbiological, chemical syntheses, chemical analyses, pharmaceutical preparation, formulation, and delivery, and treatment of patients.

As used herein, the terms “pure” and “purity” in combination with a number (e.g. 95% pure) refer to compounds and substances of the instant disclosure being present in weight/weight amount relative to other compounds and substances. For example, a composition that is 95% pure nematicidal crystal means that 95% of the composition (weight/weight) is nematicidal crystal protein and 5% of the composition (w/w) comprises one or more other substances.

As used herein, the phrase “crystal content” in combination with a percentage (e.g. 95% crystal content) refers to nematicidal crystals of the instant disclosure being present in a composition in the percentage of the total weight of the compositions. In certain embodiments, the compositions of the invention have at least 80%, at least 90%, or at least 95% (e.g., 95%, 96%, 97%, 98%, 99% or 100% crystal content.

As used herein, the term “substantially free of” in reference to a composition or compound (e.g. a compound substantially free of spores) means that the composition or compound contains less than or equal to 5% w/w of another substance (e.g. the compound has less than or equal to 5%, 2%, 1%, 0.5%, 0.1%, 0.01%, 0.001% or 0.0001% w/w of spores).

As used in this specification and the appended claims, the singular forms “a,” “an” and “the” include plural references unless the content clearly dictates otherwise. Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers. Each embodiment in this specification is to be applied mutatis mutandis to every other embodiment unless expressly stated otherwise.

As used herein, the term “about” in quantitative terms refers to plus or minus 5% of the value it modifies (rounded up to the nearest whole number if the value is not subdividable, such as a number of molecules, nucleotides, or amino acids). For example, “about 20%” would encompass 15-20% and “about 80%” would encompass 75-85%, inclusive. Moreover, where “about” is used herein in conjunction with a quantitative term it is understood that in addition to the value plus or minus 5%, the exact value of the quantitative term is also contemplated and described. For example, the term “about 23%” expressly contemplates, describes, and includes exactly 23%.

### EXAMPLES

The following Examples are presented by way of illustration and not limitation.

### Example 1: Creation of IBaCC

For production of Cry5B in sporulation-defective cells, B. thuringiensis (Bt) strain 4D8 which lacks any crystal protein expressing (Identification of Bacillus thuringiensis subsp. kurstaki strain HD1-Like bacteria from environmental and human samples after aerial spraying of Victoria, British Columbia, Canada, with Foray 48B. Appl Environ Microbiol. 2001 March; 67(3):1035-43) and in which the master spo0A regulator of sporulation (spo0A-) was deleted by homologous recombination. This composition is further referred to as Cry5B-BaCC (Bacillus with Cytosolic Crystal).

To inactivate Cry5B-BaCC, the transformed B. thuringiensis 4D8 strain was propagated aerobically in 200 mL volume in 2 liter baffled flasks with shaking at 30° C. in three-fold concentrated Luria-Bertani broth (LB) supplemented with 10 μg/mL erythromycin and 200 μg/mL kanamycin for 48 hours. The transformed B. thuringiensis cells were spun down at 4500 rpm for one hour at 4° C. and resuspended to ¼ of the original cell culture volume with prechilled sterile double-distilled water, and then were treated with 1 mg/mL carvacrol (a food-grade antimicrobial) for 15 min with shaking at 4° C. The carvacrol-treated cells were spun down and washed three times with prechilled sterile double-distilled water. Final pellets were concentrated 40 times and were stored at −80° C. until use. The dead BaCC containing biologically active Cry5B crystals are termed IBaCC (Inactivated Bacillus with Cytosolic Crystal). The 4D8 strain is also capable of autolysis, naturally lysing at the end of the growth cycle and releasing the crystal. Killing with carvacrol and homogenization can still be used to kill any residual cells and break open any residual intact IBaCC.

### Example 2: Purification of Cry5B from IBaCC Using Homogenization

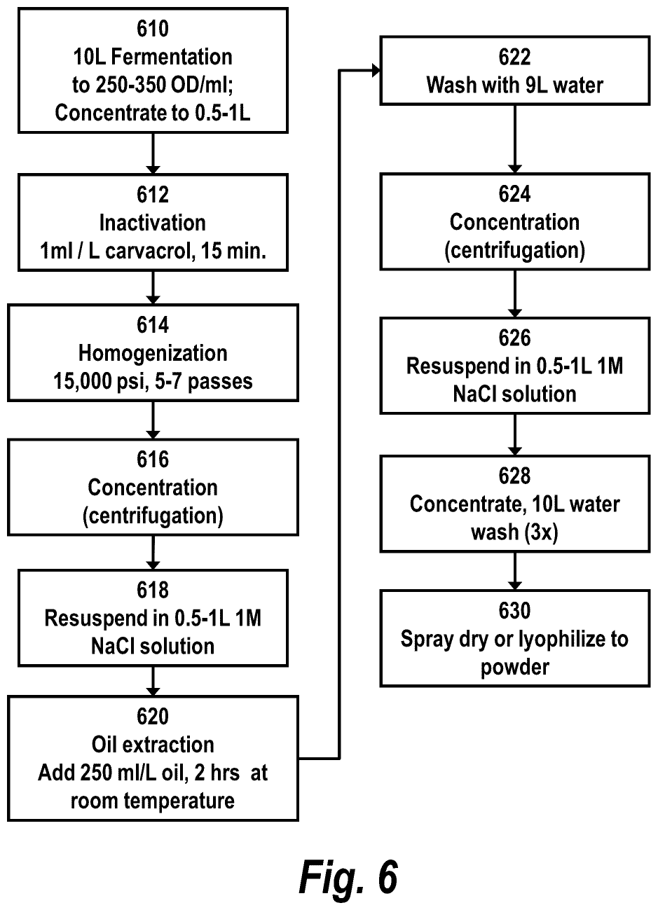

FIG. 6 shows a flow chart of the process used to purify Cry5B crystal protein from IBaCC. To purify large amounts of Cry5B protein from IBaCC, a 10 L fermentation harvest was undertaken (FIG. 6, step 610). Ten liters of BaCC were grown to between 250-350 OD/ml. The BaCC was then concentrated to between 0.5 L and 1 L using centrifugation (8000×g, 30 minutes), and then inactivated by adding 1 ml/L carvacrol and stirring for 15 minutes at room temperature (step 612) to produce IBaCC. Other methods of concentrating the BaCC prior to the inactivation step may be used, such as ultrafiltration or diafiltration.

The IBaCC was homogenized at 15,000 psi for 5 to 7 passes (step 614), and the resulting lysate was centrifuged to concentrate the bacterial lysate (step 616). Other methods of concentrating the IBaCC may be used, such as ultrafiltration or diafiltration. The resulting pellet was resuspended in 0.5 L to 1 L of 1M NaCl solution (step 618). The concentrated lysate was then mixed with 250 ml/L oil for two hours at room temperature to extract the carvacol (step 620). Following the oil extraction, 9 L of water was then added (step 622), and the mixture was again concentrated (step 624) by centrifugation. The concentrated pellet was then resuspended in 0.5 L to 1 L of NaCl solution (step 626). The mixture was then washed three times with 10 L water and concentrated (step 628) by centrifugation (8000×g, 30 minutes). The resulting composition is a purified Cry5B crystal (PCC). In some embodiments, the composition may be spray-dried or lyophilized (step 630).

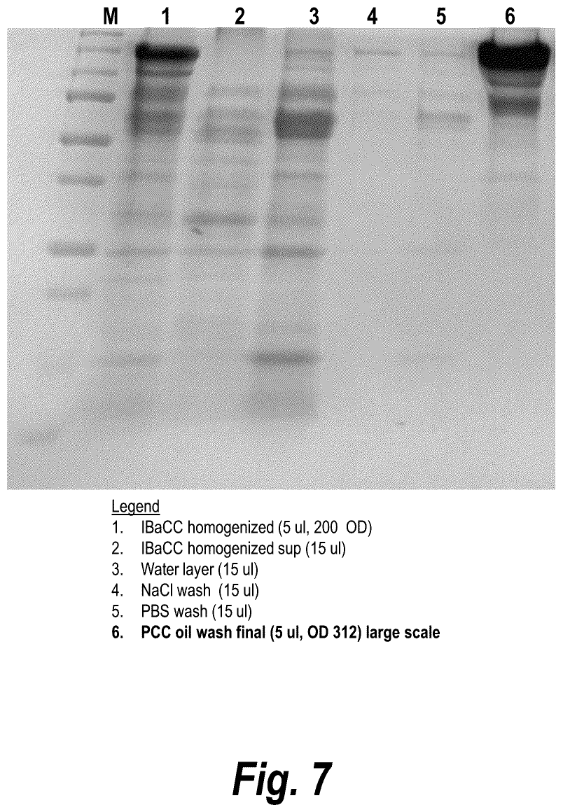

FIG. 7 shows a protein gel with samples from various stages of the purification process. Lane 1 of the gel is 5 μL of homogenized IBaCC after being grown to 200 nm OD. Lane 2 is a 15 μL sample of supernatant from concentrated homogenized IBaCC. Lane 3 is a 15 μL sample of a water layer following washing of the oil-extracted IBaCC lysate. Lane 4 is a 15 μL sample of the 1M NaCl wash (step 626). Lane 5 is a 15 μL sample of a phosphate buffered saline wash of pelleted IBaCC. Lane 6 is a 15 μL sample of PCC following step 628 (from another batch of BaCC grown to an OD of 312 Absorbance 600 units).

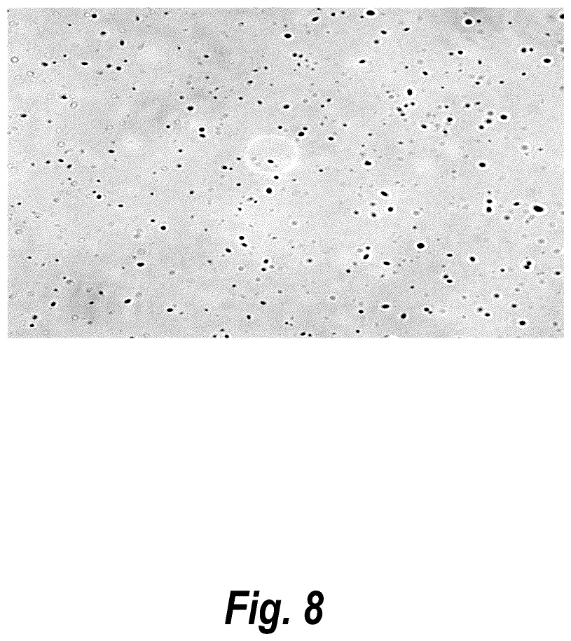

FIG. 8 is a phase-contrast photomicrograph of PCC particles at 1000× magnification.

### Example 3: Purification of Cry5B from Autolyzed IBaCC

Some strains of BaCC, such as strain 4D8, autolyse at the end of their growth cycle. This autolysis can obviate or reduce the need for a homogenization step following inactivation with an antimicrobial compound. Thus, referring to purification flow chart of FIG. 6, the homogenization step 614 may be eliminated or the number of passes reduced if an autolysing strain of bacteria is used. In such embodiments, a 10 L fermentation harvest of an autolysing BaCC may be undertaken (FIG. 6, step 610). Ten liters of BaCC may be grown to between 250-350 OD/ml. The BaCC may then be concentrated to between 0.5 L and 1 L using centrifugation, and then inactivated by adding 1 ml/L carvacrol and stirring for 15 minutes at room temperature (step 612) to produce IBaCC. Since the IBaCC have autolysed before and/or after inactivation, homogenization was not necessary. Other methods of concentrating the BaCC prior to the inactivation step may be used, such as ultrafiltration or diafiltration.

The lysate from the autolysed IBaCC may then be concentrated by centrifugation, ultrafiltration, or diafiltration (step 616). The resulting pellet may then be resuspended in 0.5 L to 1 L of 1M NaCl solution (step 618). The concentrated lysate may then be mixed with 250 ml/L oil for two hours at room temperature to extract the carvacol (step 620). Following the oil extraction, 9 L of water may then be added (step 622), and the mixture may be concentrated again (step 624) by centrifugation, ultrafiltration, or diafiltration. The concentrated mixture may then be resuspended in 0.5 L to 1 L of NaCl solution (step 626). The mixture may then be washed three times with 10 L water and concentrated (step 628) by centrifugation, ultrafiltration, or diafiltration. The resulting composition will be a purified Cry5B crystal (PCC). In some embodiments, the composition may then be spray-dried or lyophilized (step 630).

### Example 4: PCC Intoxicates Whipworms In Vitro

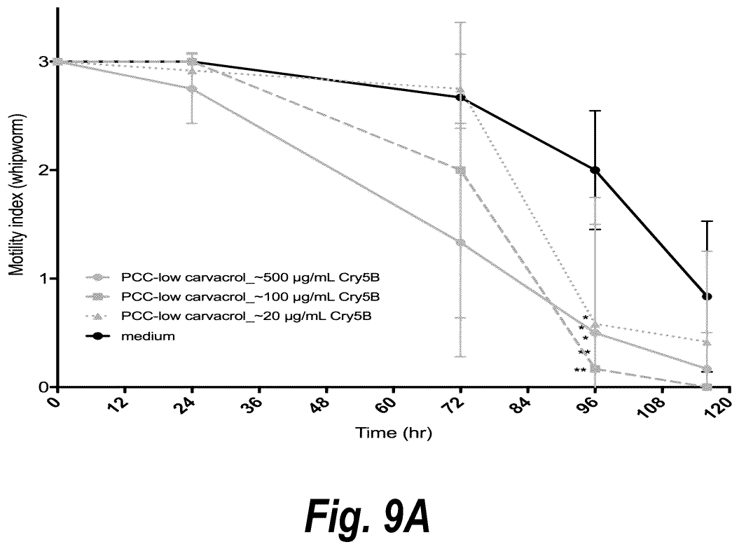

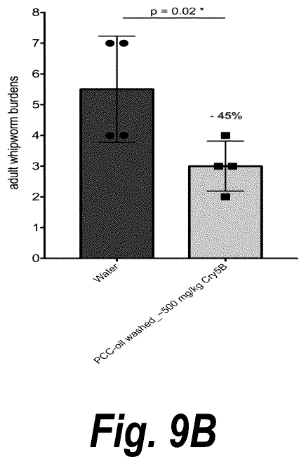

Whipworm mobility in vitro was examined following treatment with PCC made as described in Example 2. Three whipworms were added per well in RPMI medium with antibiotics, 4 wells per dose, and doses of 20, 100, and 500 μg/ml of PCC (Cry5B). FIG. 9A shows a graph of the dose-dependent effect of PCC (Cry5B) on whipworm motility measured at the times indicated. The data is expressed as the average motility index per well, where 3=highly motile, 2=slowly motile, 1=immotile unless touched, 0=immotile even when touched.

### Example 5: PCC Reduces Whipworm Burdens In Vivo

The ability of PCC to reduce whipworm burdens in hamsters was examined. Mice were infected with whipworms, Trichuris muris. Mice were split into two groups (4 mice per group), water and PCC-oil washed at −500 mg/kg of Cry5B made as described in Example 2. Both groups were administered water and PCC-oil washed via gavage in 0.5 ml volumes, single dose. Pre-treatment fecal egg counts were measured at day 34 post-infection to place the mice into two groups of equivalent fecal egg counts. Mice were treated by gavage day 35 post-infection. Post-treatment evaluation was conducted on day 41 post-infection (6 days after treatment) in which all mice were opened up and total whipworm counts taken (cecum). There was a 45% reduction in whipworm burdens (average of 5.5 whipworms in water; average of 3 whipworms in PCC), p=0.02.

FIG. 9B shows a histogram of this experiment, comparing whipworm burdens of hamsters treated with water (control) and hamsters treated with 500 mg/kg PCC made as described in Example 2.

### Example 6: PCC Intoxicates Hookworms In Vitro

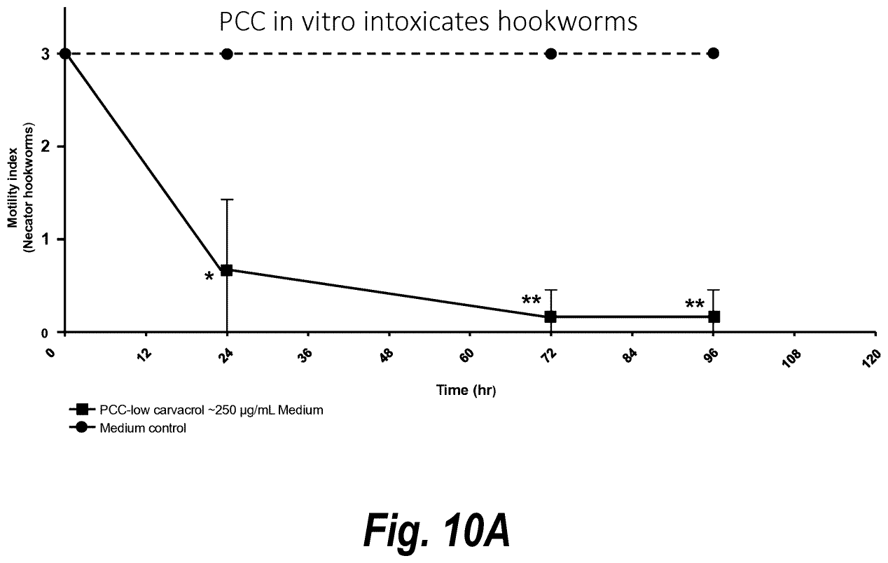

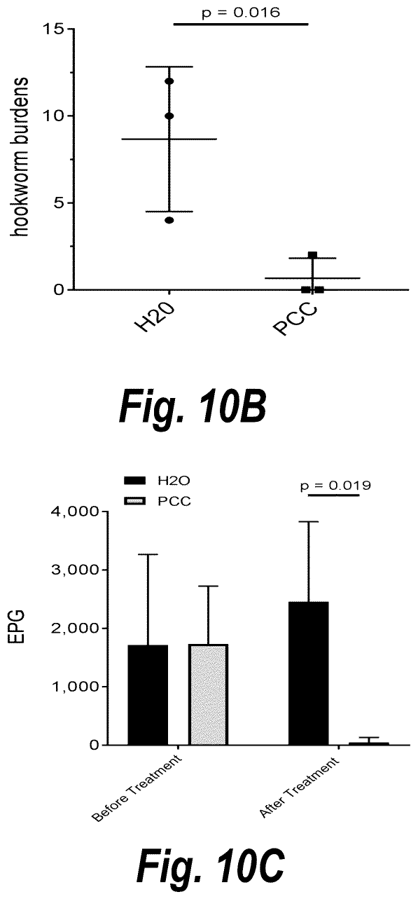

Hookworm (Necator americanus) mobility in vitro was examined following treatment with PCC made as described in Example 2. Three hookworms were added per well, 4 wells per dose, at a dose of 250 μg/ml. FIG. 10A shows a graph of changes in hookworm motility over time following in vitro exposure to doses of Cry5B PCC. PCC had a dose-dependent effect on hookworm intoxication. The data is expressed as the average motility index per well, where 3=highly motile, 2=slowly motile, 1=immotile unless touched, 0=immotile even when touched.

### Example 7: PCC Reduces Hookworm Burdens In Vivo

PCC was used at a dose of 10 mg/kg to treat hamsters infected with hookworm Necator americanus. Six hamsters were infected with N. americanus and kept on a regimen of immunosuppression (dexamethasone in the drinking water and twice weekly injections) for the entire duration of the experiment in order to prevent expulsion of the parasites. Following inoculation (approximately 60 days later) three hamsters were treated per os with a single dose of 10 mg/kg of PCC and three hamsters were treated with water as a control. Fecal egg counts were collected before treatment to determine worm loadings and hamsters were grouped into two groups of equivalent fecal egg counts prior to treatment. At 5 days post-treatment, animals were sacrificed and intestinal worm burdens determined.

FIG. 10B shows a histogram of hookworm burdens in hamsters treated with Cry5B PCC and water control. FIG. 10C shows a bar histogram of hookworm fecal egg counts in hamsters before and after treatment with Cry5B PCC and water control. A single PCC dose of 10 mg/kg resulted in a 92% drop in hookworm burden (P=0.016), and a 98% drop in fecal egg count (FEC; P=0.0-19).

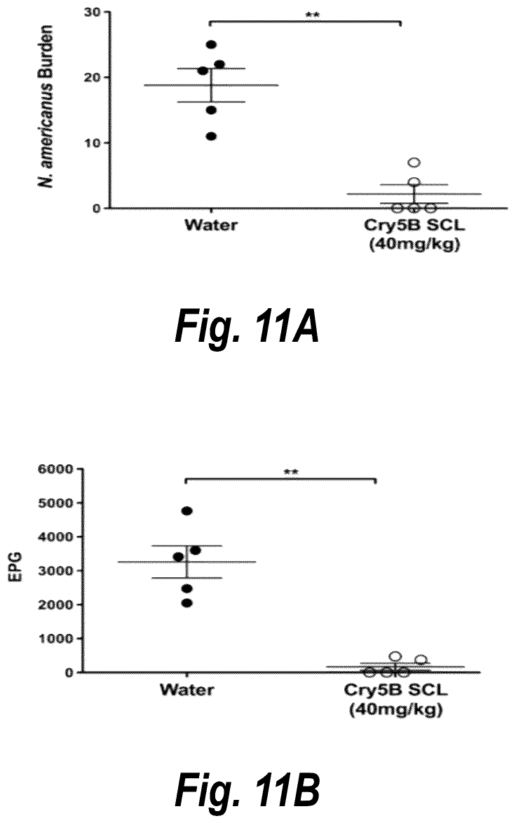

In contrast, Cry5B spore-containing lysate (SCL) requires four times the dose to achieve equivalent hookworm burden reduction in hamsters. Hamsters infected with hookworm Necator americanus and treated with a single 40 mg/kg dose of Cry5B. FIG. 11A shows a plot of hookworm burden in hamsters following treatment with either Cry5B SCL per os or water control. FIG. 11B shows a plot of eggs per gram in hamsters following treatment with either Cry5B SCL or water control. The single dose of 40 mg/kg Cry5B SCL resulted in an 88% drop in hookworm burden and a 95% drop in fecal egg count.

### Example 8: PCC is an Effective Toxin Against Hookworm Eggs

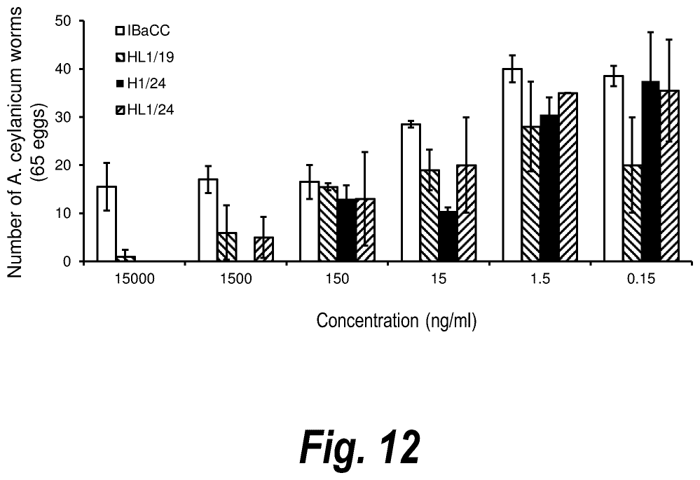

FIG. 12 shows a bar histogram of various concentrations of Cry5B PCC (three preparations) toxicity on Ancyclostoma ceylanicum hookworm egg-to-larval development compared to Cry5B IBaCC. Some of the Cry5B PCC was treated by the addition of lysozyme, which did not appear to affect the toxicity of the Cry5B PCC. A. ceylanicum eggs (˜65/well with multiple wells/dose) were placed in buffer with an E. coli food source and allowed to develop 7 days at 25C. The number of eggs that hatch and develop to the third larval infectious stage is noted at day 7. PCC is able to inhibit development of this hookworm even at low concentrations (with an IC₅₀ of −15 ng/mL) and with higher activity than IBaCC.

### Example 9: Improved Method for Purification of Purified PCC from BaCC

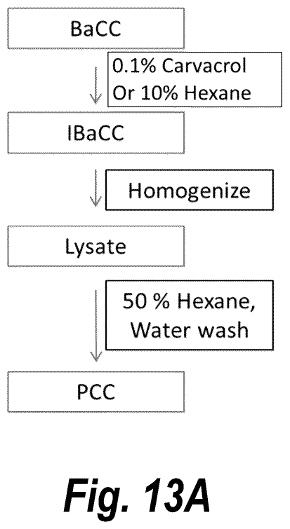

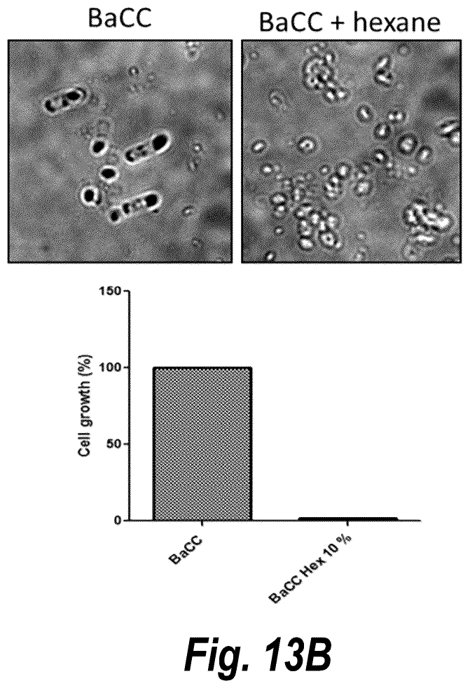

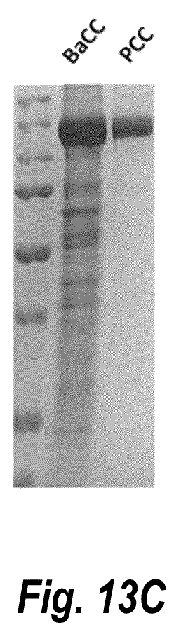

B. thuringiensis expressing Cry5B was fermented as described in Example 1, i.e., (BaCC) at the 10 L scale (Step 631) (FIG. 13A). After 10-fold cell concentration by centrifugation (4,500 rpm, 60 min at 4° C.) (Step 632), the washed BaCC was suspended in 500 ml water (final volume) and hexane was added to 10% v/v final concentration and mixed in a sealed vessel for 1 hour at room temperature to prepare IBaCC (Step 633). FIG. 13B shows microscopic images illustrating the production of IBaCC from BaCC. The microscopic image on the left shows the BaCC, and the microscopic image on the right shows the effect of hexane on BaCC, with the rod-structure destroyed. The bar graph of FIG. 13B shows the quantification of the percentage cell growth of BaCC (100% cell growth) and BaCC treated with 10% hexane (0% cell growth, complete inactivation). FIG. 13C shows an image of an SDS-PAGE gel, depicting the starting BaCC material and the final purified PCC product, following the process depicted in the diagram of FIG. 13A. Other methods of cell inactivation, e.g., surfactants, microbicides, biocides, etc., can substitute for hexane.

Purified Cry5B Crystals (PCC) were produced from IBaCC as described in Example 2. Briefly, IBaCC was homogenized at 15,000 psi for 5 to 7 passes to lyse the cells, releasing the PCC from the IBaCC cell wall ghosts (Step 634).

To purify the PCC by phase-partioning from the soluble cell components, lipids, and cell wall debris, hexane was added to 50% v/v and mixed vigorously in a sealed vessel for 1 hour at room temperature. The emulsion was transferred to sealed bottles and centrifuged at 4,500 rpm for 1 hour at 4° C. (Step 635) The upper hexane layer was collected and transferred to solvent waste. The interface layer containing cell wall debris was removed and discarded. The aqueous layer containing water-soluble cellular constituents was removed and discarded, and the PCC in the pellet was retained. Following water washing the PCC pellets were stored frozen. The resulting PCC had 98% protein w/w. Other methods of PCC recovery, such as continuous centrifugation to collect the PCC in the aqueous phase and the hexane and interface in the supernatant phase are possible. The final PCC can be stored frozen or lyophilized or spray dried and stored at room-temperature, 4° C., or frozen.

### Example 10: PCC Purified by the Improved Method is an Effective Toxin Against Nematodes

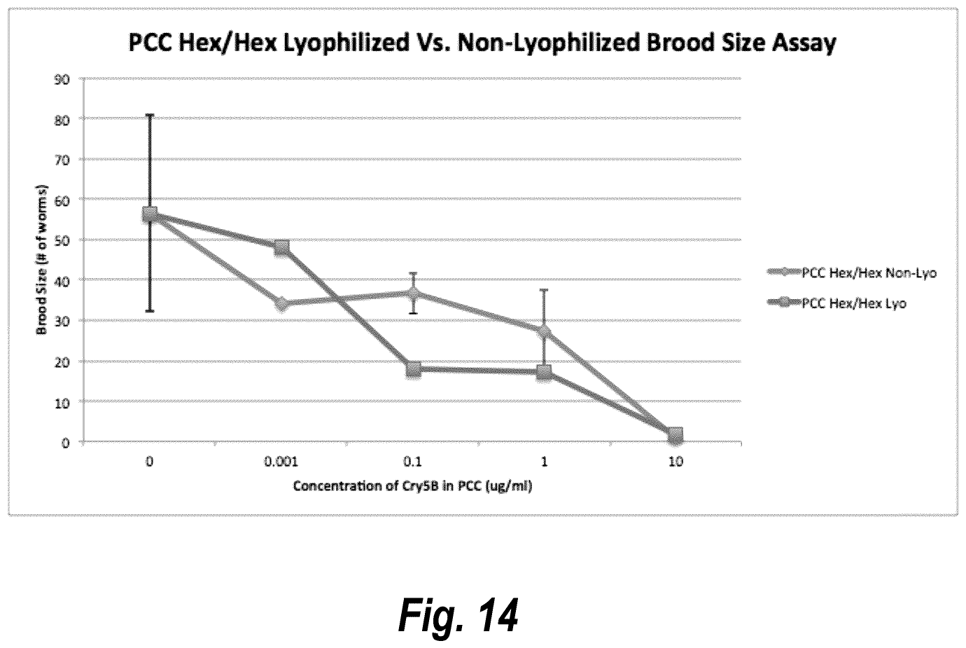

In another example, the method of Example 9 was combined with the hexane phase partitioning method of Example 9. FIG. 14 shows a graph of the bioactivity of PCC processed from BaCC with 10% hexane to kill the bacterium and 50% hexane to remove other bacterial contaminants (PCC-Hex/Hex). The non-lyophilized PCC is shown on the graph as diamonds and the lyophilized PCC is shown as squares. The readout is the average three-day brood size of a single C. elegans starting at the larval stage L4 (n=3 per condition). The PCC efficacy is excellent since at 10 μg/mL there is complete inhibition of brood size production. In comparison, published data indicates an average 70% inhibition at this dose (Hu, Y, et al., Proc Natl Acad Sci USA 107: 5955-60 (2010)). These data demonstrate that the PCC Hex/Hex process is fully compatible with lyophilization.

### Example 11: PCC Purified by the Improved Method is an Effective Toxin Against Hookworms

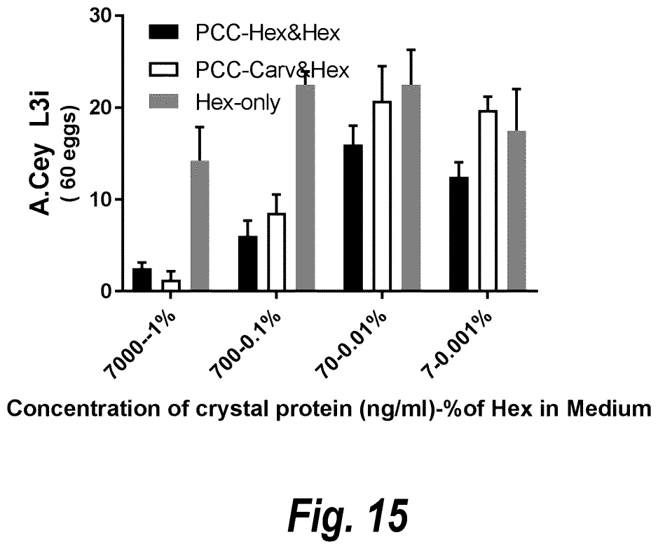

FIG. 15 shows the bioactivity of PCC processed from BaCC with carvacrol to kill the bacterium (IBaCC), and 50% hexane phase partitioning to remove other bacterial contaminants (PCC-Carv&Hex). The readout is the percent of hookworm A. ceylanicum eggs that reach mature infectious larval stage at 7 days. A lower bar indicates more inhibition of hookworm growth and development, and is indicative of intoxication. Control is hexane only in the medium at the same final concentration as in the sample with PCC. The data show that hexane alone has no effect on the parasites.

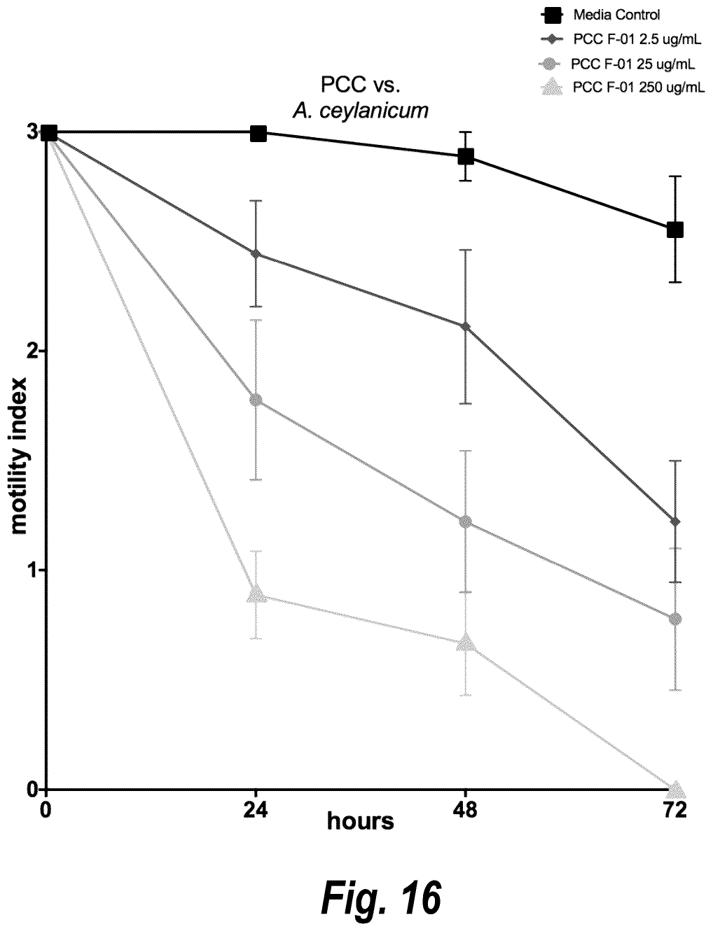

FIG. 16 shows the bioactivity of PCC processed from BaCC with carvacrol to kill the bacterium and 50% hexane to remove other bacterial contaminants. The readout is motility of hookworm A. ceylanicum adults in vitro scored every 24 hours, where 3=active and highly motile; 2=slowly motile; 1=immotile unless touched (highly intoxicated); 0=immotile even when touched (presumably dead). The data show a clear dose-response with increasing doses of PCC resulting in decreasing motility, demonstrating that the PCC processed via hexane is highly bioactive.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

While particular steps, elements, embodiments and applications of the present invention have been shown and described herein for purposes of illustration, it will be understood, of course, that the invention is not limited thereto since modifications may be made by persons skilled in the art, particularly in light of the foregoing teachings, without deviating from the spirit and scope of the invention. Accordingly, the invention is not limited except as by the appended claims.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1-81. (canceled)

82. A method of treating a parasitic worm or helminth infection in a subject comprising:
administering to the subject a therapeutically effective amount of a pharmaceutical composition comprising isolated native, bioactive nematicidal crystals formed from a single type of nematicidal crystal protein,
wherein the pharmaceutical composition is substantially free of any bacterial spores or host bacterial proteins other than nematicidal crystal protein in the form of a crystal.

83. The method of claim 82, wherein the nematicidal crystal protein is selected from the group consisting of Cry5B, Cry5C, Cry5D, Cry6A, Cry13A, Cry14A, Cry21A, Cry21B, Cry55B, and variants and truncations thereof.

84. The method of claim 82, wherein the nematicidal crystal protein is Cry5B.

85. The method of claim 82, wherein the nematicidal crystal protein is Cry5B variant Ser407Cys.

86. The method of claim 82, wherein the pharmaceutical composition further comprises a second crystal protein in the form of an isolated native, bioactive nematicidal crystal formed from only the second crystal protein.

87. The method of claim 82, wherein the pharmaceutical composition comprises at least about 95% isolated native, bioactive nematicidal crystal content.

88. The method of claim 82, wherein the pharmaceutical composition is in a dry powdered form and is encapsulated by a pharmaceutical capsule.

89. The method of claim 82, wherein the crystal protein is produced by a non-sporulating form of host bacterium.

90. The method of claim 89, wherein the non-sporulating host bacterium is a Bacillus species.

91. The method of claim 89, wherein the non-sporulating host bacterium is Bacillus thuringiensis (Bt), an E. coli species or P. flurorescens species.

92. The method of claim 89, wherein the non-sporulating host bacterium is genetically engineered to have a genetic mutation that results in a defect in sporulation such that the native, bioactive nematicidal crystal is trapped in the cytosol of the bacterium.

93. The method of claim 92, wherein the genetic mutation resulting in a defect of sporulation is the deletion or inactivation of one or more genes selected from the group consisting of: kinA, kinB, spo0A, spo0B, spo0E, spo0F, spo0J, spo0M spoIIB, spoIID, spoIIE, spoIIF, spoIIG, spoliL, spoIIM, spoIIIA, spoIIIB, spoIIIE, spoIVA, spoIVC, spoIVD, spoVG, spoVK, spoVL, spoVM, spoVN, spoVP, spoVQ, spoVID, σH, σF, σE, σG, and σK.

94. The method of claim 89, wherein the non-sporulating host bacterium is genetically engineered to express the single type of nematicidal crystal protein under the control of a non-sporulation specific promoter.

95. The method of claim 94, wherein the non-sporulation specific promoter is a Cry3A, GerA, GNAT, or TadA promoter.

96. The method of claim 89, wherein the non-sporulating host bacterium is exposed to an antimicrobial compound to inactivate the host bacterium,
optionally wherein the antimicrobial compound is iodine, a pharmaceutical antibiotic, a beta lactam antibiotic, or an organic solvent.

97. The method of claim 96, wherein a food-grade oil is added to the inactivated host bacterium to extract the antimicrobial compound, soluble cell components, lipids, and cell wall debris from the nematicidal crystal protein.

98. The method of claim 96, wherein an organic solvent is added to the inactivated host bacterium to extract the antimicrobial compound from the nematicidal crystal protein.

99. The method of claim 96, wherein the inactivated host bacterium is homogenized to form a bacterial lysate that includes the native, bioactive nematicidal crystals.

100. The method of claim 82, wherein the subject is a mammal.

101. The method of claim 82, wherein the pharmaceutical composition is suitable for oral administration.
